(19) United States
(12) Patent Application Publication
(10) Pub. No.: US 2024/0123088 A1
(43) Pub. Date: Apr. 18, 2024

(54) USE OF A SPLIT dCAS FUSION PROTEIN SYSTEM FOR EPIGENETIC EDITING

(71) Applicant: THE REGENTS OF THE UNIVERSITY OF CALIFORNIA, Oakland, CA (US)

(72) Inventors: Julian Halmai, Sacramento, CA (US); Kyle Fink, Sacramento, CA (US); Jennifer Waldo, Sacramento, CA (US)

(73) Assignee: THE REGENTS OF THE UNIVERSITY OF CALIFORNIA, Oakland, CA (US)

(21) Appl. No.: 18/490,443

(22) Filed: Oct. 19, 2023

Related U.S. Application Data
(63) Continuation of application No. PCT/US2022/025412, filed on Apr. 19, 2022.
(60) Provisional application No. 63/177,523, filed on Apr. 21, 2021.

(51) Int. Cl. A61K 48/00; C12N 9/02; C12N 9/22; C12N 9/90; C12N 15/11; C12N 15/86

(52) U.S. Cl. CPC A61K 48/005; C12N 9/0071; C12N 9/22; C12N 9/90; C12N 15/11; C12N 15/86; C12Y 114/11; C12Y 599/00; C07K 2319/09; C07K 2319/92; C12N 2750/14143; C12N 2800/40; C12N 2840/445

(57) ABSTRACT

Disclosed herein are systems, compositions and methods for using a split dCas protein system to modify the epigenetic profile of a gene of interest. The systems, compositions, and methods are useful for modifying the epigenetic profile of a particular gene within a cell, based on the discovery that effective expression of a larger-sized recombinant protein can be successfully achieved using two separate expression cassettes each encoding a half of the protein fused with a half of an intein, utilizing the unique feature of an intein system to ultimately rejoin the two halves to form one larger fusion protein with the intein spliced out.

### CROSS-REFERENCE TO RELATED APPLICATIONS

This application claims priority to, and is a 35 U.S.C. § 111(a) continuation of, PCT international application number PCT/US2022/025412 filed on Apr. 19, 2022, incorporated herein by reference in its entirety, which claims priority to, and the benefit of, U.S. provisional patent application Ser. No. 63/177,523 filed on Apr. 21, 2021, incorporated herein by reference in its entirety. Priority is claimed to each of the foregoing applications.

The above-referenced PCT international application was published as PCT International Publication No. WO 2022/225978 A1 on Oct. 27, 2022, which publication is incorporated herein by reference in its entirety.

### INCORPORATION-BY-REFERENCE OF SEQUENCE LISTING

This application includes a sequence listing in a text file entitled “UC-2021-640-2-US-seq-listing.xml” created on Oct. 19, 2023 and having a 65 kb file size. The sequence listing is submitted electronically through Patent Center and is incorporated herein by reference in its entirety.

### BACKGROUND OF THE INVENTION

Altered, especially suppressed gene expression, has been observed and revealed as a significant cause or contributing factor in a variety of diseases. The methylation status or epigenetic profile of pertinent genomic sequences is now understood as an important aspect of gene expression control. Modifying the epigenetic profile and therefore regulating the expression of a disease-relevant genomic sequence within live cells therefore presents a meaningful therapeutic approach. As such, there exists a pressing need for the development of new and effective methods for modifying the methylation status of target genes within live cells. The present invention fulfills this and other related needs.

### BRIEF SUMMARY OF THE INVENTION

This invention provides new epigenetic editing systems, compositions, and methods useful for modifying the epigenetic profile of a particular gene within a cell, based on the discovery that effective expression of a larger-sized recombinant protein can be successfully achieved using two separate expression cassettes each encoding a half of the protein fused with a half of an intein, utilizing the unique feature of an intein system to ultimately rejoin the two halves to form one larger fusion protein with the intein spliced out. Thus, in one aspect, the present invention provides an epigenetic editing system comprising: (i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an epigenetic modifier. In the alternative, the epigenetic editing system comprising: (i) a first expression cassette comprising a first polynucleotide sequence encoding an N-terminal split protein, which comprises, from its N-terminus, an epigenetic modifier, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and a transcription activator.

In some embodiments, the system further comprises a third expression cassette comprising a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site.

Alternatively, the first and/or second expression cassettes may further comprise a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of the target gene transcription start site.

In some embodiments, the system utilizes a transcription activator selected from VP64, an MS2-loop SAM system, a mini-VPR, p30000RE, and any combination thereof. In some embodiments, the dCas9 protein is a Streptococcus pyogenes dCas9 (spdCas9) protein. In some embodiments, the N-dCas9 and C-dCas9 consist of the 1 to 713 segment and the 713 to 1368 segment of SEQ ID NO:1, respectively. In some embodiments, the intein is Rhodothermus marinus (Rma) DNA helicase DnaB. In some embodiments, the N-intein and C-intein consist of the 1 to 102 segment and the 103 to 154 segment of SEQ ID NO:2, respectively. In some embodiments, the system utilizes an the epigenetic modifier selected from a human Ten-Eleven Translocation methylcytosine dioxygenase 1 catalytic domain (hTET1CD), a Suntag, a DOT1L catalytic domain, PRDM9CD, an amoeba Tet1 (NgTet1), and any combination thereof.

In some embodiments, each of the first, second, or third polynucleotide sequence is operably linked to a promoter and optionally further to a polyA sequence. One exemplary promoter is a CMV promoter. In some embodiments, the N-terminal split protein further comprises at least one nuclear localization signal (NLS) located at the N-terminus to the transcription activator (or epigenetic modifier in an alternative embodiment). In some embodiments, the C-terminal split protein further comprises at least one NLS, preferably two or three NLS, located between the C-dCas9 and the epigenetic modifier (or transcription activator in an alternative embodiment). One exemplary NLS is an SV40 NLS.

In some embodiments, the first, second, and/or third expression cassette comprises a coding sequence encoding two or three sgRNAs. In some embodiments, the first, second, and third expression cassettes are present in three separate vectors. In some embodiments, each of the vectors is a viral vector or a plasmid. Some exemplary viral vectors include lentiviral vectors, adeno-associated viral (AAV) vectors, or adenoviral vectors. In some embodiments, the system is designed to target the gene CDKL5. For example, the target sequence used may comprise or consist of the following:

(SEQ ID NO: 12)
AGAGCATCGGACCGAAGCGG,
(SEQ ID NO: 13)
GGGGGAGAACATACTCGGGG,
or
(SEQ ID NO: 14)
CCCAGGTTGCTAGGGCTTGG.

The second aspect of the present invention provides a host cell comprising the epigenetic editing system described above and herein. In some embodiments, the host cell is a mammalian cell, such as a human cell. In some embodiments, the host cell is an induced pluripotent stem cell (iPSC) or a neural stem cell (NSC).

In some embodiments, the present invention provides a host cell comprising (i) an N-terminal split protein, which comprises, from its N-terminus, a transcription activator, N-terminal half of a catalytically inactive Cas9 protein (N-dCas9), and N-terminal half of an intein (N-intein); (ii) a C-terminal split protein, which comprises, from its N-terminus, C-terminal half of the intein (C-intein), C-terminal half of the dCas9 protein (C-dCas9), and an epigenetic modifier; and (iii) at least one small guide RNA (sgRNA), each of which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site. In some embodiments, the N-terminal split protein further comprises at least one NLS located at the N-terminus to the transcription activator, and/or wherein the C-terminal split protein further comprises at least one NLS, preferably two or three NLS, located between the C-dCas9 and the epigenetic modifier.

In some embodiments, the present invention provides a host cell comprising (i) a fusion protein, which comprises, from its N-terminus, a transcription activator, N-dCas9, C-dCas9, and an epigenetic modifier; and (ii) at least one small guide RNA (sgRNA), each of which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site. In some embodiments, the fusion protein further comprises at least one NLS located at the N-terminus to the transcription activator, the at least one NLS, preferably two or three NLS, located between the C-dCas9 and the epigenetic modifier.

As an alternative, the present invention provides a host cell comprising (i) a fusion protein, which comprises, from its N-terminus, an epigenetic modifier, N-dCas9, C-dCas9, and a transcription activator; and (ii) at least one small guide RNA (sgRNA), each of which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site. In some embodiments, the fusion protein further comprises at least one NLS located at the N-terminus to the epigenetic modifier, the at least one NLS, preferably two or three NLS, located between the C-dCas9 and the transcription activator.

In some embodiments, the NLS is an SV40 NLS. In some embodiments, the epigenetic editing system, the split proteins, the fusion protein, and the sgRNA(s) are designed to target the gene CDKL5.

In a third aspect, the present invention provides a composition comprising the epigenetic editing system or the host cell as described above and herein, optionally with an excipient or a pharmaceutically acceptable carrier.

In a fourth aspect, the present invention provides a method for modulating a target gene expression in a cell, a tissue or an organ, comprising introducing into the cell/tissue/organ an effective amount of a composition comprising the epigenetic editing system described above and herein, thereby modulating the methylation profile and thus the expression of the target gene.

In some embodiments, the cell is a mammalian cell, such as a human cell, which may be a part of a tissue or an organ. In some embodiments, the cell is a neuronal cell. In some embodiments, the cell is an induced pluripotent stem cell (iPSC) or a neural stem cell (NSC). In some embodiments, the epigenetic editing system used in the method is designed to target the gene CDKL5 and increase CDKL5 gene expression in a cell, tissue, or organ that originally had a hypermethylated CDKL5 promoter and therefore suppressed CDKL5 expression, by introducing into the cell/tissue/organ an effective amount of a composition comprising the epigenetic editing system in order to increase CDKL5 gene expression.

In a fifth aspect, a method is provided for a method for treating CDKL5 deficiency disorder (CDD) in a subject in need thereof. The method includes the step of administering to the subject an effective amount of each of: (i) a first expression cassette comprising a first polynucleotide sequence encoding an N-terminal split protein, which comprises, from its N-terminus, a transcription activator, N-terminal half of a catalytically inactive Cas9 protein (N-dCas9), and N-terminal half of an intein (N-intein); and (ii) a second expression cassette comprising a second polynucleotide sequence encoding a C-terminal split proteion, which comprises, from its N-terminus, C-terminal half of the intein (C-intein), C-terminal half of the dCas9 protein (C-dCas9), and an epigenetic modifier, thereby increasing CDKL5 gene expression in the subject. In the alternative, the first expression cassette comprising a first polynucleotide sequence encoding an N-terminal split protein, which comprises, from its N-terminus, an epigenetic modifier, N-terminal half of a catalytically inactive Cas9 protein (N-dCas9), and N-terminal half of an intein (N-intein); and (ii) a second expression cassette comprising a second polynucleotide sequence encoding a C-terminal split proteion, which comprises, from its N-terminus, C-terminal half of the intein (C-intein), C-terminal half of the dCas9 protein (C-dCas9), and a transcription activator.

In some embodiments, the method further includes administering to the subject an effective amount of (iii) a third expression cassette comprising a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of CDKL5 gene transcription start site. CDKL5 gene expression in the subject.

In some embodiments, the first and/or second expression cassette further comprises a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of CDKL5 gene transcription start site.

In some embodiments, the method is practiced by administering the first, second, and/or third expression cassettes to the subject in one single composition. Alternatively, the first, second, and/or third expression cassettes are administered to the subject in two or three compositions. In some embodiments, the subject is an infant or a juvenile human. In some embodiments, the subject is an adult human.

In a sixth aspect, the present invention provides a method for treating CDKL5 deficiency disorder (CDD) in a subject in need thereof. The method includes the step of administering to the subject an effective amount or an adequate number of the host cells comprising the epigenetic editing system, expression cassettes/vectors, split proteins, fusion proteins, and sgRNA(s) described above and herein. In some embodiments, the host cells are induced pluripotent stem cells (iPSCs) or neural stem cells (NSCs). In some embodiments, the administering step comprises intravenous, intranasal, intracranial, intrathecal, or intracisternal magna administration.

### BRIEF DESCRIPTION OF THE DRAWINGS

FIG. 1 (A) Graphical representation of the different inteins used in the study. The different inteins resulted in various sized split proteins for packaging into two AAV vectors for delivery into the CNS. The most permissive backbone for a C-terminal effector domain was the small 85 kDa Rina intein fusion. (B) Western blot analysis of trans-spliced dCas9-intein protein in 293T cells. The presence of bands when stained for anti-hemagglutinin (red) and anti-FLAG (green) demonstrated the presence of N-terminal and C-terminal dCas9-split proteins. Co-transfection of pAAV-CMV-SpdCas9N and SpdCas9C resulted in the formation of a full-length dCas9 protein. No trans-splicing was detected for the Mxe and Npu inteins. Anti-actin was used as a loading control.

FIG. 2 (A) 5-methylcytosine levels in a CpG context (5meCG) over total CpG context as assessed by targeted bisulfite sequencing across 21 CpG dinucleotides in mock-treated cells or cells transfected to express trans-spliced dCas9-no effector or dCas9 fused to the catalytic domain of murine Tet1 (mTet1CD), human TET1CD with a single NLS (hTET1CD) or with three NLS (v2), a hTET1CD-SunTag or a full-length dCas9-TET1CD. The X-axis depicts the individual CpG position relative to the amplicon. (B) Mean 5-methylcytosine levels in a CpG context over all 21 CpG dinucleotides in all treatment groups.*significantly different from Mock, Tukey's HSD (p<0.05).

FIG. 3 (A) 5-methylcytosine levels in a CpG context (5meCG) over total CpG context as assessed by targeted bisulfite sequencing across 21 CpG dinucleotides in mock-treated cells or cells transfected to express trans-spliced dCas9-no effector or dCas9 fused to the catalytic domain of murine Tet1 (mTet1CD), human TET1CD with a single NLS (hTET1CD) or with three NLS (v2), a hTET1CD-SunTag or a full-length dCas9-TET1CD. The X-axis depicts the individual CpG position relative to the amplicon. (B) Mean 5-methylcytosine levels in a CpG context over all 21 CpG dinucleotides in all treatment groups. *significantly different from Mock, Tukey's HSD (p<0.05).

FIG. 4 MECP2 reactivation using a tsdCas9-TET1CD Suntag peptide repeat array. Targeted amplicon sequencing in two to three biological replicates demonstrated proof-of-principle re-activation of wild-type MECP2 from the inactive X-chromosome in RTT-NPCs. No wild-type reads were detected in mock cells (mean=0.03±0.016, N=3) relative to a Suntag without guide RNAs (mean=0.25±0.11, N=3) or guide RNA combinations 439 (mean=1.38±1.3, N=2) or 529 (mean=0.98±0.69, N=3).

FIG. 5 Intracranial injection of AAV9 split SpdCas9 into 23 week old FVB mice. (A) Animals were sacrificed 3 weeks post-injection for Western blot analysis and for IHC labeling to probe for is-dCas9 expression. (B) IHC demonstrated robust expression of AAV9 split dCas9C-turboGFP in the striatum of FVB mice 20 days post-transduction. (C) In vivo trans-splicing of SpdCas9. Expression of FLAG-tagged N-terminal or HA-tagged C-terminal split dCas9 or a combination thereof in the striatum of FVB mice was detected by Western blot. Co-transduction in the striatum results in the formation of full-length trans-spliced dCas9. (D) Quantification of band intensity demonstrates that more than 50% of protein can be trans-spliced.

FIG. 6 Multiplex CDKL5 gRNA expression using a gRNA-tRNA array. (A) An overview of the gRNA-tRNA array, showing two glycine tRNAs interspersed with the three CDKL5 guide RNAs. (B) CDKL5 upregulation by expression from individual gRNAs or by the co-transfection of a gRNA-tRNA array with a full-length dCas9-VP64. * significantly different from dCas9, Tukey's HSD (p<0.05).

FIG. 7 MECP2 does not escape from X-chromosome inactivation. (A) There was no female-male expression of MECP2 relative to the CA5B escape gene. (B) Single cell RNA expression revealed the absence of read counts from the inactive MECP2 allele, in contrast to up to 50% expression of CA5B in tissue.

FIG. 8 Clonality analysis of RTT patient derived induced pluripotent stem cells and neuronal progenitor cells carrying a 32 bp deletion in exon 5 of MECP2. (A) MECP2 allele frequency in three biological replicates of iPSC and NPCs demonstrates clonal expression of the mutant MECP2 32 bp deletion allele from the active X-chromosome. First 22 bp of the deletion are shown. No wild-type reads were detected. Alleles with a frequency >0.2% reads are shown. (B) Indel size distribution across all reads in representative iPSC and NPC samples demonstrate the presence of a 32 bp deletion and the absence of wild-type MECP2.

### DEFINITIONS

As used herein, the singular forms “a,” “an” and “the” are intended to include the plural forms as well, unless the context clearly indicates otherwise.

As used herein, the term “about,” when referring to a measurable value such as an amount or concentration and the like, is meant to encompass variations of 20%, 10%, 5%, 1%, 0.5%, or even 0.1% of the specified amount.

As used herein, the terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “adeno-associated virus” or “AAV” refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the gene editing systems, host cells, pharmaceutical compositions, vectors, and methods disclosed herein include any of the 11 or 12 serotypes, e.g., AAV2, AAV5, and AAV8, or variant serotypes, e.g., AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure.

As used herein, the term “administering” a compound or composition to a subject means delivering the compound to the subject. “Administering” includes prophylactic administration of the compound or composition (i.e., before the disease and/or one or more symptoms of the disease are detectable) and/or therapeutic administration of the composition (i.e., after the disease and/or one or more symptoms of the disease are detectable). The methods of the present technology include administering one or more compounds or agents. If more than one compound is to be administered, the compounds may be administered together at substantially the same time, and/or administered at different times in any order. Also, the compounds of the present technology may be administered before, concomitantly with, and/or after administration of another type of drug or therapeutic procedure (e.g., surgery).

As used herein, “ameliorate,” “ameliorating,” and the like, as used herein, refer to inhibiting, relieving, eliminating, or slowing progression of one or more symptoms.

As used herein, “and/or” refers to and encompasses any and all possible combinations of one or more of the associated listed items, as well as the lack of combinations when interpreted in the alternative (“or”).

As used herein, the term “aptamer” as used herein refers to single stranded DNA or RNA molecules that can bind to one or more selected targets with high affinity and specificity. Non-limiting exemplary targets include by are not limited to proteins or peptides.

As used herein, the term “Cas9” refers to a CRISPR-associated, RNA-guided endonuclease such as Streptococcus pyogenes Cas9 (spCas9) and orthologs and biological equivalents thereof. Biological equivalents of Cas9 include but are not limited to C2c1 from Alicyclobacillus acideterrestris and Cpf1 (which performs cutting functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 or dCas9, which lacks endonuclease activity. The term “split Cas9” or “split dCas9” describes the situation in which the Cas9 or dCas9 protein is split into two halves—the N- or C-terminal half (N-Cas9 or N-dCas9 and C-Cas9 or C-dCas9)—and each is fused with one of two intein moieties (N-intein and C-intein, respectively) to form two fusion proteins, which upon interacting with each other and via intein “splicing” action rejoin the C- and N-terminal halves to form a whole Cas9 or dCas9 protein. See, e.g., U.S. Pat. No. 9,074,199 B1; Zetsche et al., Nat Biotechnol. 33(2):139-42 (2015); Wright et al., PNAS 112(10) 2984-89 (2015). Exemplary N-dCas9 and C-dCas9 in the case of spdCas9 include the 1-713 segment and 713-1368 segment of SEQ ID NO:1, respectively, whereas exemplary N-intein and C-intein in the case of Rhodothermus marinus (Rma) DNA helicase DnaB include the 1-102 segment and 103-154 segments of SEQ ID NO:2, respectively. Other segments such as of the 1 to 300±10 or 20 or 50; 1 to 500±10 or 20 or 50; 1 to 700±10 or 20 or 50; 1 to 800±10 or 20 or 50; 1 to 900±10 or 20 or 50; or 1 to 1000±10 or 20 or 50, especially the 1 to 713±2, 3, 4, 5, 6, 7, 8, 9, or 10 segments of SEQ ID NO:1 may serve as the N-dCas9. The 300±10 or 20 or 50 to 1368; 500±10 or 20 or 50 to 1368; 700±10 or 20 or 50 to 1368; 800±10 or 20 or 50 to 1368; 900±10 or 20 or 50 to 1368; or 1000±10 or 20 or 50 to 1368, especially the 713±2, 3, 4, 5, 6, 7, 8, 9, or 10 to 1368 segments of SEQ ID NO:1 may serve as the C-dCas9. The 1 to 40±5 or 10, 1 to 60±5 or 10, 1 to 80±5 or 10, 1 to 100±5 or 10, or 1 to 120±5 or 10 segments of SEQ ID NO:2 may serve as the N-intein. The 40±5 or 10 to 154, 60±5 or 10 to 154, 80±5 or 10 to 154, 100±5 or 10 to 154, or 120±5 or 10 to 154 segments of SEQ ID NO:2 may serve as the C-intein.

As used herein, the term “cell” or “host cell” may refer to either a prokaryotic or eukaryotic cell, optionally obtained from a subject or a commercially available source. Exemplary host cells include mammalian cells, especially human cells, which may be somatic cells or stem cells, such as neuronal cells or induced pluripotent stem cell (iPSCs) or neural stem cells (NSCs).

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359, and Hsu et al., Cell 156(6): 1262-1278 (2014).

As used herein, the term “comprising” is intended to mean that the compositions and methods include the recited elements, but do not exclude others.

As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

As used herein, the term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the route of administration, and the physical delivery system in which it is carried.

In some embodiments, “effective amount” or “therapeutically effective amount” refers to a quantity sufficient to achieve a desired therapeutic and/or prophylactic effect, e.g., an amount which results in the full or partial amelioration of disease or disorders or symptoms associated with mitochondrial dysfunction, neurological disease, lack of energy, glycolytic process dysfunction or cellular respiration related dysfunction in a subject in need thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. A person of ordinary skill in the art will be able to determine appropriate dosages depending on these and other factors. The compositions can also be administered in combination with one or more additional compounds. Multiple doses may be administered. Additionally or alternatively, multiple therapeutic compositions or compounds may administered. In the methods described herein, the compounds may be administered to a subject having one or more signs or symptoms of a disease or disorder described herein.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “endonuclease” refers to any suitable endonuclease enzyme protein or a variant thereof that will be specifically directed by the selected guide polynucleotide to enzymatically knock-out the target sequence of the guide polynucleotide.

As used herein, the term “variant thereof,” as used with respect to an endonuclease, refers to the referenced endonuclease in its enzymatically functional form expressed in any suitable host organism or expression system and/or including any modifications to enhance the enzymatic activity of the endonuclease.

In some embodiments of the present disclosure, a suitable endonuclease includes a CRISPR-associated sequence 9 (Cas9) endonuclease or a variant thereof, a CRISPR-associated sequence 13 (Cas13) endonuclease or a variant thereof, CRISPR-associated sequence 6 (Cas6) endonuclease or a variant thereof, a CRISPR from Prevotella and Francisella 1 (Cpf1) endonuclease or a variant thereof, or a CRISPR from Microgenomates and Smithella 1 (Cms1) endonuclease or a variant thereof. In some embodiments of the present disclosure, a suitable endonuclease includes a Streptococcus pyogenes Cas9 (SpCas9), a Staphylococcus aureus Cas9 (SaCas9), a Francisella novicida Cas9 (FnCas9), or a variant thereof. Variants may include a protospacer adjacent motif (PAM) SpCas9 (xCas9), high fidelity SpCas9 (SpCas9-FIF1), a high fidelity SaCas9, or a high fidelity FnCas9.

In some embodiments of the present disclosure, the endonuclease comprises a Cas fusion nuclease comprising a Cas9 protein or a variant thereof fused with a Fok1 nuclease or variant thereof. Variants of the Cas9 protein of this fusion nuclease include a catalytically inactive Cas9 (e.g., dead Cas9 or dCas9). In some embodiments of the present disclosure, the endonuclease may be a Cas9, Cas13, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM1 0 152, Rhodococcus erythropolis PR4, Rhodococcus jostii RFIA1, Rhodococcus opacus B4 (uid36573), Acidothermus cellulolyticus 11B, Arthrobacter chlorophenolicus A6, Kribbella flavida (DSM 17836, uid43465), Thermomonospora curvata (DSM431 83), Bifidobacterium dentium Bd1, Bifidobacterium longum DJO10A, Slackia heliotrinireducens (DSM 20476), Persephonella marina EX H1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei1 9 1, uncultured Termite group 1 bacterium phylotype Rs D 17, Fibrobacter succinogenes S85, Bacillus cereus (ATCC 10987), Listeria innocua, Lactobacillus casei, Lactobacillus rhamnosus GG, Lactobacillus salivarius UCC1 18, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS1 0565, Streptococcus gallolyticus UCN34 (uid46061), Streptococcus gordonii Challis subst CH1, Streptococcus mutans NN2025 (uid46353), Streptococcus mutans, Streptococcus pyogenes M 1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS61 80, Streptococcus pyogenes MGAS31 5, Streptococcus pyogenes SSI-1, Streptococcus pyogenes MGAS1 0750, Streptococcus pyogenes NZ1 3 1, Streptococcus thermophiles CNRZ1 066, Streptococcus thermophiles LMD-9, Streptococcus thermophiles LMG 1831 1, Clostridium botulinum A3 Loch Maree, Clostridium botulinum B Eklund 17B, Clostridium botulinum Ba4 657, Clostridium botulinum F Langeland, Clostridium cellulolyticum H 10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus moniliformis (DSM 121 12), Bradyrhizobium BTAil, Nitrobacter hamburgensis X14, Rhodopseudomonas palustris BisB1 8, Rhodopseudomonas palustris BisB5, Parvibaculum lavamentivorans DS-1, Dinoroseobacter shibae DFL 12, Gluconacetobacter diazotrophicus Pal 5 FAPERJ, Gluconacetobacter diazotrophicus Pal 5 JGI, Azospirillum B51 0 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 8 1116, Campylobacter jejuni, Campylobacter lari RM21 00, Helicobacter hepaticus, Wolinella succinogenes, Tolumonas auensis DSM 9 187, Pseudoalteromonas atlantica T6c, Shewanella pealeana (ATCC 700345), Legionella pneumophila Paris, Actinobacillus succinogenes 130Z, Pasteurella multocida, Francisella tularensis, Francisella novicida U112, Francisella tularensis holarctica, Francisella tularensis FSC 198, Francisella tularensis, Francisella tularensis WY96-3418, or Treponema denticola (ATCC 35405).

As used herein, the term “epigenetic modifier” encompasses any enzyme or a portion thereof capable of catalyzing the methylation or demethylation of a DNA sequence at one or more CpG islands so as to alter the methylation profile of the DNA sequence.

As used herein, the terms “equivalent” and “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “expression cassette” refers to a DNA construct that comprises at least one coding sequence operably linked to a promoter that directs the transcription of the coding sequence, which may encode an mRNA sequence that is ultimately translated to a protein or may encode an RNA molecule that does not translate to a protein but rather exerts its function in the form of an RNA molecule (e.g., as an sgRNA or siRNA or tRNA). Optionally the “expression cassette” further includes elements such as a transcription termination element and a polyA signal. In some cases, each “expression cassette” may include two or more coding sequences, which may be controlled by one single promoter or by separate promoters. Additional transcription regulatory elements may be included in the “expression cassette” as well. An “expression cassette” can take the form of a linear or circular DNA molecule, such as a plasmid or a viral vector.

As used herein, the term “functional” may be used to modify any molecule, biological, or cellular material to intend that it accomplishes a particular, specified effect.

As used herein, the term “guide polynucleotide” refers to a polynucleotide having a “synthetic sequence” capable of binding the corresponding endonuclease enzyme protein (e.g., Cas9) or a protein comprising the fusion of a transcription activator, a DNA epigenetic modifier, and an enzymatically inactive Cas protein (dCas), artificially joined together from two split proteins by way of intein “splicing” mechanism and a variable target sequence capable of binding the genomic target (e.g., a nucleotide sequence found in an exon of a target gene such as CDKL5). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

As used herein, “hybridization” or “hybridizes” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6× saline-sodium citrate (“SSC”) to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M sodium chloride (“NaCl”) and 15 mM citrate buffer. It is understood that equivalents of SSC using other buffer systems can be employed.

As used herein, the term “isolated” as used herein refers to molecules or biologicals or cellular materials being substantially free from other materials.

As used herein, the term “lentivirus” refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy—Tools and Potential Applications,” ISBN 978-953-51-1014-9, DOI: 10.5772/52534.

As used herein, the terms “nucleic acid sequence,” “nucleotide sequence,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “organ” a structure which is a specific portion of an individual organism, where a certain function or functions of the individual organism is locally performed and which is morphologically separate. Non-limiting examples of organs include the skin, blood vessels, cornea, thymus, kidney, heart, liver, umbilical cord, intestine, nerve, lung, placenta, pancreas, thyroid and brain.

As used herein, the term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

As used herein, “prevention,” “prevents,” or “preventing” of a disorder or condition refers to a compound that, in a statistical sample, reduces the occurrence of the disorder, symptom, or condition in the treated sample relative to a control subject, or delays the onset of one or more symptoms of the disorder or condition relative to the control subject.

As used herein, the term “promoter” as used herein refers to any sequence that regulates the expression of a coding sequence, such as a gene. refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CDKL5 promoter, SCML2 promoter, COL9A3 promoter, MECP2, CMV promoter and U6 promoter, the phosphoglycerate kinase 1 (PGK) promoter; SSFV, CMV, MNDU3, SV40, Ef1a, UBC and CAGG. Non-limiting exemplary promoter sequences are provided herein below:

CMV promoter (SEQ ID NO:9) ATACGCGTTGACATTGATTATTGACTAGTTATTAATAGTAATCAATTACGGGGTC ATTAGTTCATAGCCCATATATGGAGTTCCGCGTTACATAACTTACGGTAAATGGC CCGCCTGGCTGACCGCCCAACGACCCCCGCCCATTGACGTCAATAATGACGTATG TTCCCATAGTAACGCCAATAGGGACTTTCCATTGACGTCAATGGGTGGAGTATTT ACGGTAAACTGCCCACTTGGCAGTACATCAAGTGTATCATATGCCAAGTACGCCC CCTATTGACGTCAATGACGGTAAATGGCCCGCCTGGCATTATGCCCAGTACATGA CCTTATGGGACTTTCCTACTTGGCAGTACATCTACGTATTAGTCATCGCTATTACC ATGGTGATGCGGTTTTGGCAGTACATCAATGGGCGTGGATAGCGGTTTGACTCAC GGGGATTTCCAAGTCTCCACCCCATTGACGTCAATGGGAGTTTGTTTTGGCACCA AAATCAACGGGACTTTCCAAAATGTCGTAACAACTCCGCCCCATTGACGCAAATG GGCGGTAGGCGTGTACGGTGGGAGGTCTATATAAGCAGAGCTCGTTTAGTGAAC CGTCAGATCGCCTGGAGACGCCATCCACGCTGTTTTGACCTCCATAGAAGACACC GGGACCGATCCAGCCTCCGGACTCTAGAGGATCGAACCCTT, or a biological equivalent thereof.

U6 promoter (SEQ ID NO:10) GAGGGCCTATTTCCCATGATTCCTTCATATTTGCATATACGATACAAGGCTGTTA GAGAGATAATTAGAATTAATTTGACTGTAAACACAAAGATATTAGTACAAAATA CGTGACGTAGAAAGTAATAATTTCTTGGGTAGTTTGCAGTTTTAAAATTATGTTTT AAAATGGACTATCATATGCTTACCGTAACTTGAAAGTATTTCGATTTCTTGGCTTT ATATATCTTGTGGAAAGGACGAAACACC, or a biological equivalent thereof.

A number of effector elements are disclosed herein for use in these vectors; e.g., a tetracycline response element (e.g., tetO), a tet-regulatable activator, T2A, VP64, RtA, KRAB, and a miRNA sensor circuit. The nature and function of these effector elements are commonly understood in the art and a number of these effector elements are commercially available. Non-limiting exemplary sequences thereof are disclosed herein and further description thereof is provided herein below.

As used herein, the term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “protospacer adjacent motif” (PAM) refers to a short nucleotide sequence adjacent to a target sequence (protospacer) that is recognized (targeted) by a sgRNA/Cas endonuclease system (including split Cas or split dCas system) described herein. The sequence and length of a PAM herein can differ depending on the Cas/dCas protein or Cas/dCas fusion protein used. The PAM sequence can be of any length but is typically 1, 2, 3, 4, 5, 6, 7, 8, 9, 10, 11, 12, 13, 14, 15, 16, 17, 18, 19 or 20 nucleotides long. The PAM sequence plays a key role in target recognition by licensing sgRNA base pairing to the protospacer sequence (Szczelkun et al., Proc. Natl. Acad. Sci. U.S.A. 111: 9798-803 (2014)).

As used herein, the term “recombinant expression system” refers to a system comprising two or more genetic constructs (expression cassettes, e.g., in the form of viral vectors such as AAV vectors) for the expression of polynucleotide sequences formed by recombination, for example, the coding sequences for an N-terminal split protein, a C-terminal split protein, and/or for sgRNA.

As used herein, the term “sgRNA” or “single guide RNA” as used herein refers to the guide RNA sequences used to target specific genes for correction employing the CRISPR technique. Techniques of designing sgRNAs and donor therapeutic polynucleotides for target specificity are well known in the art. For example, Doench et al., Nature Biotechnology 32(12):1262-7 (2014), Mohr et al., FEBS J. 283: 3232-38 (2016), and Graham et al., Genome Biol. 16:260 (2015). sgRNA comprises or alternatively consists essentially of, or yet further consists of a fusion polynucleotide comprising CRISPR RNA (crRNA; i.e., a scaffold region) and trans-activating CRIPSPR RNA (tracrRNA; i.e., a spacer region); or a polynucleotide comprising crRNA (i.e., a scaffold region) and tracrRNA (i.e., a spacer region). In some aspects, an sgRNA is synthetic (Kelley et al., J of Biotechnology 233:74-83 (2016).

As used herein, the term “subject,” “individual,” or “patient” may refer to an individual organism, a vertebrate, a mammal, or a human. “Mammal” includes a human, non-human mammal, non-human primate, murine (e.g., mouse, rat, guinea pig, hamster), ovine, bovine, ruminant, lagomorph, porcine, caprine, equine, canine, feline, avis, etc. In any embodiment herein, the mammal is feline or canine. In any embodiment herein, the mammal is human, who may be an adult (at least 18 years of age) or a juvenile (younger than 18 years of age).

As used herein, “target sequence” refers to a nucleotide sequence adjacent to a 5′-end of a protospacer adjacent motif (PAM). Being “adjacent” herein means being within 1 to 8 nucleotides of the site of reference, including being “immediately adjacent,” which means that there is no intervening nucleotides between the immediately adjacent nucleotide sequences and the immediately adjacent nucleotide sequences are within one nucleotide of each other.

As used herein, “target site” refers to a site of the target sequence including both the target sequence and its complementary sequence, for example, in double stranded nucleotides. The target site described herein may mean a nucleotide sequence hybridizing to a sgRNA spacer region, a complementary nucleotide sequence of the nucleotide sequence hybridizing to a sgRNA spacer region, and/or a nucleotide sequence adjacent to the 5′-end of a PAM. Full complementarity of a sgRNA spacer region with a target site is not necessarily required, provided there is sufficient complementarity to cause hybridization and promote formation of a CRISPR complex. A target sequence or target site may comprise any polynucleotide, such as DNA or RNA polynucleotides. In some embodiments, a target sequence or target site is located in the nucleus or cytoplasm of a cell. In some embodiments, the target sequence or target site may be within an organelle of a eukaryotic cell, for example, mitochondrion or chloroplast.

As used herein, the term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, the term “transcription activator” encompasses any protein or a portion thereof capable of initiating or enhancing the transcription of a genomic DNA sequence from a nearby transcription start site, e.g., within about 1,000 base pairs or about 500 base pairs or about 200 or 100 base pairs, of where the “transcription activator” is localized (e.g., bound) to the DNA sequence.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention or prophylaxis.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell.

A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) The Promise of Stem Cells for Neural Repair, Brain Res. Vol. 1091(1):258-264; Pluchino et al. (2005) Neural Stem Cells and Their Use as Therapeutic Tool in Neurological Disorders, Brain Res. Brain Res. Rev., Vol. 48(2):211-219; and Goh, et al. (2003) Adult Neural Stem Cells and Repair of the Adult Central Nervous System, J. Hematother. Stem Cell Res., Vol. 12(6):671-679.

As use herein, the term “differentiation” describes the process whereby an unspecialized cell acquires the features of a specialized cell such as a heart, liver, or muscle cell. “directed differentiation” refers to the manipulation of stem cell culture conditions to induce differentiation into a particular cell type. “Dedifferentiated” defines a cell that reverts to a less committed position within the lineage of a cell. As used herein, the term “differentiate,” including any of its grammatical variations, defines a cell that takes on a more committed (i.e., “differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal. Conversely, “dedifferentiated” describes a cell that reverts to a less committed position within the lineage of a cell. Induced pluripotent stem cells are examples of dedifferentiated cells.

As used herein, the “lineage” of a cell defines the heredity of the cell, i.e., its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques.

Another type of vector is a viral vector, wherein virally-derived DNA or RNA sequences are present in the vector for packaging into a virus (e.g., retroviruses, replication defective retroviruses, adenoviruses, replication defective adenoviruses, lentiviruses, replication defective lentiviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous viral expression vectors include retroviruses, replication defective retroviruses, adenoviruses, replication defective adenoviruses, lentiviruses, replication defective lentiviruses, and adeno-associated viruses.

It is to be inferred without explicit recitation and unless otherwise intended, that when the present disclosure relates to a polypeptide, protein, polynucleotide or antibody, a fragment an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, or a hydroxyl group(s) it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

### DETAILED DESCRIPTION OF THE INVENTION

I. General Methodology

Embodiments according to the present disclosure are described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting. Throughout and within this disclosure various technical and patent publications are references by a citation or an Arabic numeral. The full bibliographic citations for each reference identified by an Arabic numeral is found in the reference section, immediately preceding the claims.

It is to be appreciated that certain aspects, modes, embodiments, variations and features of the present methods are described below in various levels of detail in order to provide a substantial understanding of the present technology. The definitions of certain terms as used in the specification are provided below. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. The term consisting of intends the recited elements and any additional elements that do not materially change of the function of the recited element or elements.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The practice of the present technology employs, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

II. Epigenetic Editing Systems

The disclosure provides an epigenetic editing system comprising, or alternatively consisting essentially of, or yet further alternatively consisting of: (i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an epigenetic modifier. The N-terminal split protein and the C-terminal split protein, upon their production and by way of intein “splicing” mechanism, ultimately become rejoined to form the fusion protein having the three main components of transcription activator-dCas9-epigenetic modifier. In the alternative, the first expression cassette comprising a first polynucleotide sequence encoding an N-terminal split protein, which comprises, from its N-terminus, an epigenetic modifier, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and a transcription activator. The N-terminal split protein and the C-terminal split protein, upon their production and by way of intein “splicing” mechanism, ultimately become rejoined to form the fusion protein having the three main components of epigenetic modifier-dCas9-transcription activator.

In some embodiments, the system further includes a third expression cassette comprising a third polynucleotide sequence encoding at least one small guide RNA (sgRNA), optionally two or three sgRNAs, each of which comprises, or consists essentially of, or consisting of a scaffold region and a spacer region, wherein the spacer region hybridizes to a sequence complementary to a target sequence adjacent to 5′ end of a protospacer adjacent motif (PAM), with both the target sequence and the PAM located within 1 kilobase (kb) of a target gene (e.g., the CDKL5 gene) transcription start site.

In some embodiments, the scaffold region is a sequence that is necessary for dCas9 binding to the gRNA (addgene.org/guides/crispr/). In some embodiments, the spacer region hybridizes to a nucleotide sequence that is complementary to a target sequence adjacent to a 5′-end of a protospacer adjacent motif (PAM). In some embodiments, the target sequence and the PAM are located at least about 2 or about 1 kilobase (kb), at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb from the transcriptional start site (TSS) of a target gene, e.g., the CDKL5 gene. While the target sequence and the PAM are in one aspect located can be located at least about 1 kb from the transcriptional start site, it is apparent to the skilled artisan that other ranges are within the scope of this invention, e.g., the target sequence and the PAM are located from about 2 kb, or from about 1 kb to about 0.1 kb.

In the alternative, instead of being present in a third expression cassette, the third polynucleotide sequence encoding one or more sgRNA may be included in the first expression cassette. The transcription of the third polynucleotide sequence may be directed by the same or a separate promoter used in the transcription of the first polynucleotide sequence. Similarly, the third polynucleotide sequence encoding one or more sgRNA may be included in the second expression cassette, either instead of the first expression cassette or in addition to the first expression cassette.

In some embodiments, the first polynucleotide sequence encodes an N-terminal split protein having (from the N-terminus) the main components of a transcription activator, an N-dCas9, and an N-intein, whereas the second polynucleotide sequence encodes a C-terminal split protein having (from the N-terminus) the main components of a C-intein, a C-dCas9, and an epigenetic modifier. An exemplary dCas9 protein is a catalytically inactive Streptococcus pyogenes dCas9 (spdCas9) protein, which may be split into 1 to 713(±1, 2, 3, 4, 5, 6, 7, 8, 9, 10, 15, 20, 25, 30, 40, 50, 60, 70, 80, 90, or 100) and 713(±1, 2, 3, 4, 5, 6, 7, 8, 9, 10, 15, 20, 25, 30, 40, 50, 60, 70, 80, 90, or 100) to 1368 segments of SEQ ID NO:1 (dCas9 protein sequence) as N-dCas9 and C-dCas9, respectively. An exemplary intein is Rhodothermus marinus (Rma) DNA helicase DnaB, which may be split into 1 to 102(±1, 2, 3, 4, 5, 6, 7, 8, 9, 10, 12, 15, 20, 25, or 30) and 103(±1, 2, 3, 4, 5, 6, 7, 8, 9, 10, 12, 15, 20, 25, or 30) to 154 segments of SEQ ID NO:2 (Rma intein sequence) as N-intein and C-intein, respectively.

In some embodiments, the transcription activator comprises VP64 or a biologically active fragment of VP16. Transcription factors act through a DNA-binding domain that localizes a protein to a specific site within the genome and through accessory effector domains that either activate or repress transcription at or near that site. Effector domains, such as the activation domain the herpes simplex virus VP16 (Morgan L Maeder et al., (2013) Nat Biotechnol. 31(12):1137-42) and the repression domain Krüppel-associated box (KRAB), are modular and retain their activity when they are fused to other DNA-binding proteins. In some embodiments, VP64 is the activation domain VP16. In some embodiments. VP64 is a recombinant tetrameric repeat of comprising the minimal activation domain VP64. In some embodiments, the activation domain of VP16 comprises amino acids 413-489 of the VP16 protein. In some embodiments, the recombinant tetrameric repeat of VP16's minimal activation domain comprises, or consists essentially of, or yet further consists of the amino acid sequence DALDDFDLDIVIL (SEQ ID NO:11). In some embodiments, the transcription activator includes one or more of VP64, VP64-p65-Rta triparte fusion (addgene.org/99670/), and SunTag. SunTag is a novel protein scaffold/tagging system with a repeating peptide array for signal amplification in gene expression.

An exemplary transcription activator includes one or more of VP64, an MS2-loop SAM system, a mini-VPR, and p30000RE. An exemplary epigenetic modifier includes one or more of Ten-Eleven Translocation methylcytosine dioxygenase 1 catalytic domain (TET1CD), such as a human TET1CD (hTET1CD), a Suntag, p30000RE, a DOT1L catalytic domain, PRDM9CD, and an amoeba Tea (NgTet1).

In some embodiments, each of the first, second, or third polynucleotide sequence is operably linked to a promoter, for example, a CMV promoter. Optionally, the first and/or second polynucleotide sequences are further operably linked to a polyA sequence.

In some embodiments, either or both of the N-terminal split protein and the C-terminal split protein further comprise at least one nuclear localization signal (NLS) in order to facilitate the nuclear translocation of the ultimate fusion protein (transcription activator-dCas9-epigenetic modifier). For example, one, two or three NLS sequences may be placed at the N-terminus to the transcription activator in the N-terminal split protein. NLS sequences may be placed directly adjacent to the N-terminus of the transcription activator or may be separated from the N-terminus of the transcription activator by an intervening amino acid or peptide sequence. Also, when more than one NLS sequence is used, all NLS sequences may be placed directly adjacent to one another, each NLS sequence may be separated by an intervening amino acid or peptide sequence, or a combination thereof. Another suitable location for placing the NLS sequence(s) is between the C-dCas9 and the epigenetic modifier in the C-terminal split protein. One exemplary NLS is an SV40 NLS.

A third polynucleotide sequence may be present in the DNA epigenetic editing system of this invention, either separately from the first and second polynucleotide sequences (i.e., in a third expression cassette) or together with one or both of the first and second polynucleotide sequences (i.e., in the same expression cassette or cassettes), and encodes for at least one small guide RNA (sgRNA) for the purpose of guiding the (transcription activator-dCas9-epigenetic modifier) fusion protein ultimately joined from the N-terminal split protein and the C-terminal split protein to effectuate changes in DNA epigenetic profile (i.e., methylation or demethylation of CpGs or GC islands or region) at positions of at least about −1500, at least about −1000, at least about −500, at least about −200, at least about −148, at least about −66 and, at least about −19 relative to transcription start site of the target genomic sequence.

In some embodiments, each of the first, second, and third polynucleotide sequences is carried by a separate expression vector. Alternatively, the third polynucleotide sequence may be present in the same vector along with the first or second polynucleotide sequence. A commonly used expression vectors may be a plasmid or a viral vector, for example, a lentiviral vector, an adeno-associated viral (AAV) vector, or an adenoviral vector.

In some embodiments, the viral vector is selected from the group of retroviral vectors, adenovirus vectors, adeno-associated virus vectors, or alphavirus vectors. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof. Further details as to modern methods of vectors for use in gene transfer may be found in, for example, Kotterman et al. (2015) Viral Vectors for Gene Therapy: Translational and Clinical Outlook Annual Review of Biomedical Engineering 17. In some embodiments, the viral vector is a selected from the group of a lentiviral vector, an adeno-associated viral (AAV) vector, or an adenoviral vector. In some embodiments, the viral vector is a lentiviral vector. In some embodiments, the lentiviral vector is an optimized lentiviral sgRNA cloning vector with MS2 loops at tetraloop and stemloop 2 and EF1a-puro resistance marker.

In some embodiments, the first nucleotide and second nucleotide molecules permit the transcriptional reprogramming of a gene promoter by precisely demethylating gene promoters or enhancers for desired gene targets. Thus, in one aspect, as described herein, is a method for transcriptionally reprogramming a gene promoter in a cell in need thereof, by inserting into the cell, the system as disclosed herein. In some embodiments, DNA is methylated at 5-cytosine (5mC), and such methylation silence gene expression and is important for genomic imprinting, regulation of gene expression, chromatic architecture organization, and cell-fate determination. In some embodiments, gene demythylation is associated with gene activation and occurs either via passive demethylation or through the oxidation of the methyl group. In some embodiments, demethylation via oxidation is mediated by TET (ten-eleven translocation) dioxygenases that oxidizes 5 methyl cytosine (5mC) to 5-hydroxymethylcytosine (5-hmC), which is a critical step in the ultimate removal of the methyl group.

In some embodiments, the full-length TET1 protein, which comprises typical features of 2OG-Fe(II) oxygenases, including conservation of residues predicted to be important for coordination of the cofactors Fe(II) and 2OG, serves as the epigenetic modifier. The full-length TET1 protein has 2136 amino acids, and comprises an N-terminal a helix followed by a continuous series of β strands, typical of the double-stranded β helix (DSBH) fold of the 2OG-Fe(II) oxygenases, a unique conserved cysteine-rich region (amino acids 1418-1610 of the full-length human TET1 protein; MIM:607790; ENSG00000138336) that is contiguous with the N terminus of the DSBH region (amino acids 1611-2074), a CXXC-type zinc-binding domain (amino acids 584-624 of the full-length human TET1 protein) domain, binuclear Zn-chelating domain, and three bipartite nuclear localization signals (NLS) (Morgan L Maeder et al., (2013) Nat Biotechnol. 31(12):1137-42; Mamta Tahiliani et al., (2009) Science 324(5929): 930-935). In some embodiments, TET1 catalytic domain (TET1CD) serves as the epigenetic modifier, which comprises, or consists essentially of, or consisting of amino acids 1418 to 2136 of the full-length TET1 protein, and encompasses the conserved cysteine-rich region and the DSBH domain (Mamta Tahiliani et al., (2009) Science 324(5929): 930-935). In some embodiments, the DSBH domain of the catalytic domain construct comprises a nuclear localization (NLS) sequence. In some embodiments, the DSBH domain of the catalytic domain construct does not comprise a NLS sequence.

In some embodiments, the (transcription activator-dCas9-epigenetic modifier) fusion protein facilitates the targeted demethylation of a target gene and induces transcription as well as expression of the gene. In particular, the fusion protein facilitates the targeted demethylation of gene targets selected from the group consisting of CDKSL, SCML2 (Sem Polyeomb Group Protein Like 2), COL9A3, or Methyl-CpG Binding Protein 2 (MECP). In some embodiments, both the first and second expression cassettes comprising the first and second polynucleotide sequences encoding the N-terminal and C-terminal split proteins and a third polynucleotide sequence encoding at least one small guide RNA (sgRNA), are required to target the (transcription activator-dCas9-epigenetic modifier) fusion protein to a specific locus to modify the epigenetic profile of a predetermined genomic DNA sequence (e.g., methylate or demethylate the genomic DNA) without altering the DNA sequence.

In some embodiments, the dCas9 is a catalytically inactive Cas9 nuclease from the Clustered regularly interspaced palindromic repeats (CRISPR), a type II bacterial adaptive immune system that has been modified to target the dCas9 to a desired genomic loci using sequence-specific guide RNAs for genome editing. In some embodiments, the desired genomic loci include any genes, optionally CDKSL, SCML2 (Scm Polycomb Group Protein Like 2), COL9A3, or Methyl-CpG Binding Protein 2 (MECP). In some embodiments, CDKL5 sgRNAs 20-bp spacer sequences are selected within at least about about 1 kb or about 2 kb, at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb of the CDKL5 TSS (chrX:18,443,725, hg19) using the CRISPR/Cas9 and TALEN online tool for genome editing, CHOPCHOP. In some embodiments, guide RNAs (sgRNAs) span DNase I hypersensitive sites and H3K4me3 peaks of the CDKL5 promoter within at least about 2 kb, at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb of window on either side of the CDKL5 transcriptional start site. In some embodiments, the third polynucleotide sequence encoding at least one small guide RNA (sgRNA) used to create target-specific sgRNA expression vectors are listed in Table 1 (SEQ ID NOs:18-52).

In some embodiments, the targeted sequence is a sequence in the gene promoter. The targeted sequence or a fragment thereof hybridizes to the corresponding gRNA. In one embodiment, the targeted sequence hybridizes to the corresponding gRNA without any mismatches. In another embodiment, the targeted sequence hybridizes to the corresponding gRNA with 1, 2, 3, 4, 5, 6, 7, 8, 9, 10 or more mismatches. Based on the targeted sequence, the gRNA sequence can be determined. In one embodiment, a gRNA comprises, or consists essentially of, or yet further consists of a sequence complement to a targeted sequence, such as those as disclosed herein, or an equivalent that is capable of binding to the same targeted sequence but comprises 1, 2, 3, 4, 5, 6, 7, 8, 9, 10 or more mismatches. In another embodiment, a gRNA comprises, or consists essentially of, or yet further consists of a sequence reverse-complement to a targeted sequence, such as those as disclosed herein, or an equivalent that is capable of binding to the same targeted sequence but comprises 1, 2, 3, 4, 5, 6, 7, 8, 9, 10 or more mismatches. In yet another embodiment, a gRNA comprises, or consists essentially of, or yet further consists of a sequence reverse to a targeted sequence, such as those as disclosed herein, or an equivalent that is capable of binding to the same targeted sequence but comprises 1, 2, 3, 4, 5, 6, 7, 8, 9, 10 or more mismatches.

In some embodiments, gene activation requires several sgRNAs. In some embodiments, gene activation requires six sgRNAs. In some embodiments, gene activation requires at least about, 1-10, 1-5, 1-6, 1-3, 3-6, or 4-6 sgRNAs. In some embodiments, the target sequence for the sgRNA comprises or consists essentially of or consist of one or more of: AGAGCATCGGACCGAAGCGG (SEQ ID NO:12), GGGGGAGAACATACTCGGGG (SEQ ID NO:13), CCCAGGTTGCTAGGGCTTGG (SEQ ID NO:14), ATCGCCTGAAACTTGTCCGG (SEQ ID NO:15), CGAAAGGGTGTGAAAGAGGG (SEQ ID NO:16), and/or TGGGGAAGGTAAAGCGGCGA (SEQ ID NO:17). In some embodiments, the target sequence for the sgRNA comprises or consists essentially of or consist of AGAGCATCGGACCGAAGC (SEQ ID NO:12). In some embodiments, the target sequence for the sgRNA comprises or consists essentially of or consist of GGGGGAGAACATACTCGGGG (SEQ ID NO:13). In some embodiments, the target sequence for the sgRNA comprises or consists essentially of or consist of

(SEQ ID NO: 14)
CCCAGGTTGCTAGGGCTTGG.

In one aspect, the present disclosure provides a third polynucleotide sequence, which may be present in the first and/or second expression cassette with the first and/or second polynucleotide sequence (e.g., in a shared vector or in a different vector) encoding a sgRNA. In some embodiments, the sgRNA comprises, or consists essentially of, or consists of a scaffold region and a spacer region. In some embodiments, the spacer region hybridizes to a nucleotide sequence that is complementary to a target sequence comprising, or consisting essentially of, or consisting of any one or more of the following sequences GGGGGAGAACATACTCGGGG (SEQ ID NO:13), AGAGCATCGGACCGAAGCGG (SEQ ID NO:12), CCCAGGTTGCTAGGGCTTGG (SEQ ID NO:14), ATCGCCTGAAACTTGTCCGG (SEQ ID NO:15), CGAAAGGGTGTGAAAGAGGG (SEQ ID NO:16), and TGGGGAAGGTAAAGCGGCGA (SEQ ID NO:17). In some embodiments, the spacer region hybridizes to a nucleotide sequence that is complementary to a target sequence comprising, or consisting essentially of, or yet further consisting GGGGGAGAACATACTCGGGG (SEQ ID NO:13). In some embodiments, the spacer region hybridizes to a nucleotide sequence that is complementary to a target sequence comprising, or consisting essentially of, or yet further consisting AGAGCATCGGACCGAAGCGG (SEQ ID NO:12). In some embodiments, the spacer region hybridizes to a nucleotide sequence that is complementary to a target sequence comprising, or consisting essentially of, or yet further consisting CCCAGGTTGCTAGGGCTTGG (SEQ ID NO:14). In some embodiments, the spacer region hybridizes to a nucleotide sequence that is complementary to a target sequence comprising, or consisting essentially of, or yet further consisting ATCGCCTGAAACTTGTCCGG (SEQ ID NO:15). In some embodiments, the spacer region hybridizes to a nucleotide sequence that is complementary to a target sequence comprising, or consisting essentially of, or yet further consisting CGAAAGGGTGTGAAAGAGGG (SEQ ID NO:16). In some embodiments, the spacer region hybridizes to a nucleotide sequence that is complementary to a target sequence comprising, or consisting essentially of, or yet further consisting

(SEQ ID NO: 17)
TGGGGAAGGTAAAGCGGCGA.

In some embodiments, the third polynucleotide sequence comprises or consists essentially of or consist of the coding sequences for at least three sgRNAs. In some embodiments, the third polynucleotide sequence encoding at least one small guide RNA (sgRNA) comprises a first sgRNA, a second sgRNA, and a third sgRNA. In some embodiments, the target sequence for the first sgRNA comprises, or consists essentially of, or consist of AGAGCATCGGACCGAAGCGG (SEQ ID NO:12). In some embodiments, the target sequence for the second sgRNA comprises, or consists essentially of, or consist of the sequence of GGGGGAGAACATACTCGGGG (SEQ ID NO:13). In some embodiments, the target sequence for the third sgRNA comprises, or consists essentially of, or consist of CCCAGGTTGCTAGGGCTTGG (SEQ ID NO:14). In some embodiments, the target sequence for the first sgRNA comprises, or consists essentially of, or consist of one or more of AGAGCATCGGACCGAAGCGG (SEQ ID NO:12), GGGGGAGAACATACTCGGGG (SEQ ID NO:13), and/or CCCAGGTTGCTAGGGCTTGG (SEQ ID NO:14).

In one aspect, the present disclosure provides a epigenetic editing system comprising, or consisting essentially of, or consisting of: (A) a first and second expression cassettes comprising a first and second polynucleotide sequences encoding an N-terminal split protein and a C-terminal split protein, which are to ultimately for a fusion protein of (transcription activator-dCas9-epigenetic modifier) to facilitate the targeted epigenetic modification of a target gene and activation of its transcription/expression (for example, selected from the group consisting of CDKSL, SCML2, COL9A3, or MECP), and (B) a third polynucleotide sequence encoding at least one single guide RNA (sgRNA), comprising, or consisting essentially of, or yet further consisting of a scaffold region and a spacer region; wherein the spacer region hybridizes to a nucleotide sequence complementary to a target gene sequence adjacent to a 5′-end of a protospacer adjacent motif (PAM); and wherein the target sequence and the PAM are located within about 2 or about 1 kilobase (kb) and ranges as described herein of the transcriptional start site (TSS) of the target gene (for example, the cyclin dependent kinase-like 5 or CDKL5 gene), and wherein the target sequence for the first sgRNA comprises or consists essentially of AGAGCATCGGACCGAAGCGG (SEQ ID NO:12), the target sequence for the second sgRNA comprises or consists essentially of or consists of GGGGGAGAACATACTCGGGG (SEQ ID NO:13), and the target sequence for the third sgRNA comprises or consists essentially of or consists of CCCAGGTTGCTAGGGCTTGG (SEQ ID NO:14). In some embodiments, the spacer region comprises, or consists essentially of, or yet further consists of a spacer sequence provided in Table 1 (SEQ ID NOs:18-52).

III. Host Cells and Compositions

The present disclosure provides an isolated or engineered host cell comprising any one or more of the epigenetic editing system, expression cassettes, expression vectors, and/or any one or more of the split proteins, fusion proteins, or sgRNAs as disclosed herein. In some embodiments, the host cell produces the epigenetic editing system, the expression cassettes and/or the vectors encoding the split proteins and sgRNA(s). Additionally or alternatively, the host cell is an insect cell, a mammalian cell, or a bacterial cell. In some embodiment, the host cell is selected from a stem cell, an embryonic stem cell (that in one aspect is from an established cultured cell line), a progenitor cell, an induced pluripotent stem cell (IPSC), a neuronal progenitor cell, a neuronal stem cell, or a stem or progenitor cell with the ability to differentiate into a neuron. The host cell can also be an egg, a sperm, a zygote, or a germline cell. In yet a further embodiment, the host cell is a human cell. In one aspect, the cell is a culture or primary cell from a human or non-human host or subject. In one aspect, the cell is a cell in need of genetic correction, e.g., a cell with suppressed expression of a gene due to improper epigenetic status of the gene, as described herein. In a further aspect, the cell is a neuronal cell with dysfunctional gene expression, e.g., due to improper epigenetic status of the gene, especially due to improper hypermethylation. The cells are useful in cell assay systems and therapies as described herein.

In some embodiments, the epigenetic editing system is engineered to yield a fusion protein of (transcription activator-dCas9-epigenetic modifier) or (epigenetic modifier-dCas9-transcription activator), which specifically targets one or more of the chromosome(s) or chromosome sites of the host cell. In some instances, the epigenetic editing system is engineered to yield a fusion protein that comprises both a transcription activator and an epigenetic modifier, with one located at the N-terminal and the other located at the C-terminal to the dCas9 portion of the fusion. In some embodiments, the host cell comprises homozygous polynucleotide sequences at the target site(s). In another embodiment, the host cell comprises heterozygous polynucleotide sequences at the target site(s). In some aspects and/or embodiments of the disclosure herein, the first and second expression cassettes are engineered to yield an ultimate fusion protein targeting one or more of the chromosome(s) or chromosome site(s) of the mammalian cell, especially a human cell.

In some embodiments, the host cell comprises gene editing systems comprising, or alternatively consisting essentially of, or yet further consisting of: (i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an epigenetic modifier. In some embodiments, the host cell further comprises a third expression cassette comprising a third polynucleotide sequence encoding at least one small guide RNA (sgRNA), optionally two or three sgRNAs, each of which comprises a scaffold region and a spacer region. While the first and second expression cassettes are typically presented in two separate expression vectors, the third expression cassette may be present in a third expression vector or may be present in a shared expression vector with the first or second expression cassette. The spacer region of the sgRNA(s) hybridizes to a nucleotide sequence that is complementary to a target sequence adjacent to a 5′-end of a protospacer adjacent motif (PAM). In some embodiments, the target sequence and the PAM are located at least 1 kilobase (kb) from the transcriptional start site (TSS) of a target gene (e.g., the CDKL5 gene).

In some embodiments, the host cell comprises the N-terminal split protein and the C-terminal split protein. In some embodiments, the host cell comprises the final fusion protein having the major components of transcription activator, dCas9, and epigenetic modifier following the rejoining event mediated by the intein. In either case, the host cell typically further comprises the sgRNA or sgRNAs. The (transcription activator-dCas9-epigenetic modifier) fusion protein targets and modifies the methylation status of the pre-determined target gene in the host cell chromosome(s), for example, induces DNA demethylation of CpGs (GC islands or region) at positions of at least about −1500, at least about −1000, at least about −500, at least about −200, at least about −148, at least about −66 and, at least about −19 base pairs relative to transcription start site of the target gene. In some embodiment, the fusion protein upregulates the expression level of the target gene in the host cell in an unmethylated chromatin context. In some embodiments, the presence of both the transcription activator and the epigenetic modifier shows a synergistic effect resulted in a greater than 60% expression of an inactive allele (i.e., silenced allele, for example, due to hypermethylation of the promoter region) in the host cell. In some embodiments, expression of the (transcription activator-dCas9-epigenetic modifier) fusion protein results in the fewest number of differentially expressed genes in RNAseq analysis.

In a related aspect, the present disclosure provides for a pharmaceutical composition comprising isolated or engineered host cells comprising any one or more of the epigenetic editing system, expression cassettes/vectors, or the split proteins or the final (transcription activator-dCas9-epigenetic modifier) or (epigenetic modifier-dCas9-transcription activator) fusion protein in addition to the one or more sgRNAs. The composition may further comprise, in addition to the host cells, other therapeutic agents, and at least one carrier, optionally one or more physiologically/pharmaceutically acceptable carriers or excipients. In some embodiments, the methylation profile of the target gene(s) on the chromosome(s) of the host cells is altered according the design of the epigenetic editing system of this invention.

The expression cassettes/vectors, the epigenetic editing system, and the host cells can be used as in vitro assays or systems to test new therapies and assess their potential efficacy. Thus, in one aspect, provided herein is a method for increasing the expression of a gene such as a CDKL5 gene expression in a cell, comprising introducing into the cell the vectors or gene editing systems as described above. In one aspect, the gene expression is increased due to reduced DNA methylation in the CDKL5 promoter region. Although CDKL5 is used as an example of such as system, one skill in the art can apply the principles of this system to other genes wherein DNA methylation is reduced, and/or the promoter region is located on a silenced X-chromosomal allele of the cell. The cells can be samples isolated from subjects suspected of containing defective gene expression and/or a commercially available or laboratory generated cell line. The host cell can be a prokaryotic or a eukaryotic cell, non-limiting examples of such include an insect cell, a mammalian cell (such as a human cell), or a bacterial cell. In some embodiments, the host cell is selected from an egg, a sperm, a zygote, or a germline cell. In yet a further embodiment, the host cell is a mammalian cell, including a human or non-human cell. In one aspect, the cell is a cell in need of genetic correction, e.g., a neuronal cell with dysfunctional gene expression, as described herein. One of skill of the art can generate the host cell system with a cell or cells from a subject according to the present invention and then test a proposed new therapeutic agent to determine whether it is effective for treating a pertinent condition. In additional or alternatively, multiple therapeutic agents may be tested for efficacy of a combination therapy strategy. Such assay system may be used in settings for in vitro, ex vivo, or in vivo studies, including as an animal model.

IV. Therapeutic Applications

The present disclosure provides an epigenetic editing system comprising two expression cassettes encoding two split proteins, which ultimately rejoin via intein “splicing” function to yield a fusion protein comprising a transcription activator, a dCas9 protein, and an epigenetic modifier. The fusion protein is then guided by at least one small guide RNA (sgRNA) for targeting a nucleotide complementary sequence located within about 1 kilobase of the transcription start site (TSS) of a target gene (such as the CDKL5 gene), thereby modifying the methylation status of the gene, especially at the promoter region and altering the expression of the gene.

A significant number of X-linked genes escape from X chromosome inactivation and are associated with a distinct epigenetic signature. One epigenetic modification that strongly correlates with X-escape is reduced DNA methylation in promoter regions. The present inventors created a new and improved artificial escape system capable of editing DNA methylation on the promoter of CDKL5, a gene causative for an infantile epilepsy, from the silenced X-chromosomal allele in human neuronal-like cells. The artificial system produces a fusion protein of a transcription activator, a dCas9 protein, and an epigenetic modifier that is capable of targeting to the CDKL5 promoter using three small guide RNAs. This artificial system can cause significant reactivation of the inactive CDKL5 allele in combination with removal of methyl groups from CpG dinucleotides. This newly improved artificial system employs a multi-vector system to address the practical difficulties associated with the larger size of a fusion protein and provides great potential for treating those suffering from X-linked disorders.

In particular, defects in epigenetics modification of ions channel in the nervous system are linked to Rett syndrome (RTT) and cyclin-dependent kinase-like 5 (CDKL5) deficiency disorder (CDD). RTT and CDKL5 deficiency disorder are two X-linked developmental brain disorders with overlapping but distinct phenotypic features. Mutations in the X-linked gene encoding methyl-CpG-binding protein 2 (MECP2) account for 90-95% of the case of classic Rett syndrome, and mutations in the X-linked gene encoding CDKL5 account from some cases of atypical RTT that manifest with early refractory epilepsy.

The neurodevelopmental disorder CDKL5 deficiency is caused by de novo mutations in the CDKL5 gene on the X chromosome (Kalscheuer et al., (2003) Disruption of the serine/threonine kinase 9 gene causes severe X-linked infantile spasms and mental retardation. Am. J. Hum. Genet., 72, 1401-1411). Due to random XCI, females affected by the disorder form a mosaic of tissue with cells expressing either the mutant or wild-type allele (Weaving et al. (2004) Mutations of CDKL5 cause a severe neurodevelopmental disorder with infantile spasms and mental retardation. Am. J. Hum. Genet., 75, 1079-1093). A potential therapeutic approach might be to activate the silenced wild-type CDKL5 allele in cells expressing the loss-of-function mutant allele. The present inventors synthetically induced escape of CDKL5 from the inactive X chromosome in the neuronal-like cell line SH-SY5Y via DNA methylation editing of the CDKL5 promoter using a VP64-dCas9-TET1 fusion protein for targeted DNA demethylation. This artificial system/synthetic induction of CDKL5 escape from XCI, resulted in a significant increase in allele-specific expression of the inactive CDKL5 allele and correlated with a significant reduction in methylated CpG dinucleotides in the CGI core promoter.

As such, the disclosure demonstrates that loss of DNA methylation is crucial for inducing escape from inactive chromosomal regions (e.g., regions of the X chromosome) and illustrates a novel therapeutic avenue for subjects suffering from or at risk of disorders (e.g., X-linked disorders) that may be prevented or treated via removal of methylation within genetic regions that are associated with the disorders. A method is disclosed for increasing CDKL5 gene expression in a cell or a subject in need thereof by introducing into the cell a pharmaceutical composition comprising an epigenetic editing system or by administering to the subject a pharmaceutical composition comprising (1) the epigenetic editing system or (2) host cells comprising the epigenetic editing system so as to reduce DNA methylation in the CDKL5 promoter region or replace cells with suppressed CDKL5 expression with modified cells that have the normal CDKL5 promoter methylation level and therefore normal CDKL5 expression level. In some embodiments, the CDKL5 promoter region is located on a silenced X-chromosomal allele of the subject. In some embodiments, the subject in need for increasing CDKL5 gene expression has been diagnosed with CDKL5 deficiency disorder (CDD). In some embodiments, the subject is a mammal, including a human or a non-human, and including a fetus, an infant, a juvenile, and an adult.

In some embodiments, the system or pharmaceutical composition or modified host cells are administered to the subject by one or more of the following means: intravenous administration, intranasal administration, intracranial administration, intrathecal administration, or intracisternal magna administration.

V. Kits

The invention also provides kits for practicing the present invention of epigenetic editing. The kit comprises, or consists essentially of, or consists of any one or more of the epigenetic editing system, the expression vectors encoding the N-terminal and C-terminal split proteins plus the sgRNA(s), the host cells, and the corresponding compositions, as well as an optional instruction for use in modifying the methylation profile of a target gene (such as activating a silenced X-chromosomal allele) in a subject in need thereof. In some embodiments, the kit is used for increasing CDKL5 gene expression in a subject in need thereof. In some embodiments, the kit is used for treating or preventing CDD in a subject in need thereof.

### VI. Examples

The following examples are provided by way of illustration only and not by way of limitation. Those of skill in the art will readily recognize a variety of non-critical parameters that could be changed or modified to yield essentially the same or similar results.

Introduction

In the last decade, viral vectors, in particular adeno-associated virus (AAV), have emerged as a promising delivery modality for gene therapies. Recently, two AAV-based gene therapies have been approved by the Federal Drug Administration, including Luxturna to treat an inherited form of blindness and Zolgensma for spinal muscular atrophy, with many more AAV-based gene therapies in clinical trials for heritable disorders concerning the blood, muscle, heart and other neurological indications(1). However, size limitations of the AAV genome reduce the packaging size of transgenes and non-coding elements such as inverted terminal repeats, promoters, post-transcriptional regulatory elements and poly(A) signals to about 4.9 kb(2). Indeed, AAV genome size has been found to be inversely correlated with viral titer and transduction efficiency, which severely hampers delivery of larger proteins in preclinical studies(1).

One protein that has revolutionized basic and translational science alike is the Streptococcus pyogenes Cas-associated protein 9 (SpCas9)(3-6). The bacterial derived nuclease is targeted by a customizable guide RNA to the intended locus in the genome and has enabled researchers to introduce insertions and deletions, insert small epitopes or larger transgenes into the host cell genome(3-6), make precise single base pair substitutions(7) or serve as a nickase(8) or DNA binding domain by using nuclease deficient SpdCas9, that allows fusion and recruitment of base editors(9), transcriptional activators(8, 10, 11), repressors(12) and epigenetic effector domains(13). However, delivery of Cas9 in an AAV vector for translational application is limited due to the large size (4.2 kb) of the transgene. Early crystallography studies have demonstrated that SpCas9 consists of two separate polypeptide chains comprised of the NIX and REC lobes that when co-delivered in vitro can be reconstituted by the guide RNA(14). While functional, the reconstituted Cas9 ribonucleoprotein was found to elicit strongly reduced DNA cleavage activity (15). This 2015 study paved the way for more elegant and functional means shortly after to deliver split versions of the Cas9 protein. To date the most well-studied means to reconstitute SpCas9 has been by inteins(16).

Inteins, similar to RNA introns, get trans-spliced out of the extein-forming polypeptide, fusing the separate polypeptides together, thereby only leaving a small amino acid scar behind. Similar to SpCas9, inteins are proteins that have been harnessed from the bacterial kingdom, such as the Nostoc punctiforme (Npu) DNA Polymerase III DnaE(17), Rhodothermus marinus (Rma) DNA helicase Dna9(18) and Mycobacterium xenopi (Mxe) Gyrase A(19). SpdCas9 can then be divided across two plasmids and fused to a N-intein and C-intein that will then find each other in the host cell upon translation of each protein piece, significantly increasing the AAV packaging capacity(17-19).

Targeted epigenetic editing of a gene on the X-chromosome was previously demonstrated. This required the co-delivery of two large SpdCas9 proteins fused to a VP64 trans-activator and the DNA demethylase TET1CD, largely extending the packaging size of individual AAV particles and making this approach an excellent target for a split SpdCas9 strategy. In addition, this approach required the simultaneous targeting of the gene promoter using three guide RNAs(20). The size constraints of AAV are further increased by the necessity to express multiple CRISPR gRNAs from separate RNApolIII promoters, such as U6 or H1(21). One additional way proposed to overcome this difficulty is to utilize a multiplex editing platform, in which the different gRNAs are expressed from the same promoter and then are post-transcriptionally cleaved into separate gRNAs(22). Therefore, a system is applied that was previously demonstrated in yeast(22) via the endogenous tRNA processing system, utilizing human glycine tRNA interspersed with gRNAs, creating a gRNA-tRNA array that allows for the expression of mature gRNAs post-processing. In this study it is demonstrated that the Rma split SpdCas9 effectively reconstitutes in vitro and in Vivo.

Robust DNA demethylation of a gene implicated in a childhood epilepsy disease, CDKL5(23), and programmable transcription in human cell lines using dual VP64 and TET1 fusion split proteins can be achieved. Finally, patient-derived induced pluripotent stem cell (iPSC) lines have been obtained from a Rett syndrome (RTT)(24) patient from Coriell, harboring a 32 bp deletion in MECP2. Expression analysis reveals that RTT iPSCs and neural stem cells (NSCs) exhibit clonality and that transfection of a targeted trans-spliced SpdCas9 DNA demethylase in the promoter is followed by mild gene reactivation. Gene regulation strategies, previously demonstrated to result in gene reactivation in neuronal-like cells, can be applied to patient-derived NSCs and will allow for the rapid development of this platform technology.

Results

In Vitro Reconstitution of Rma-Intein Trans-Spliced SpdCas9

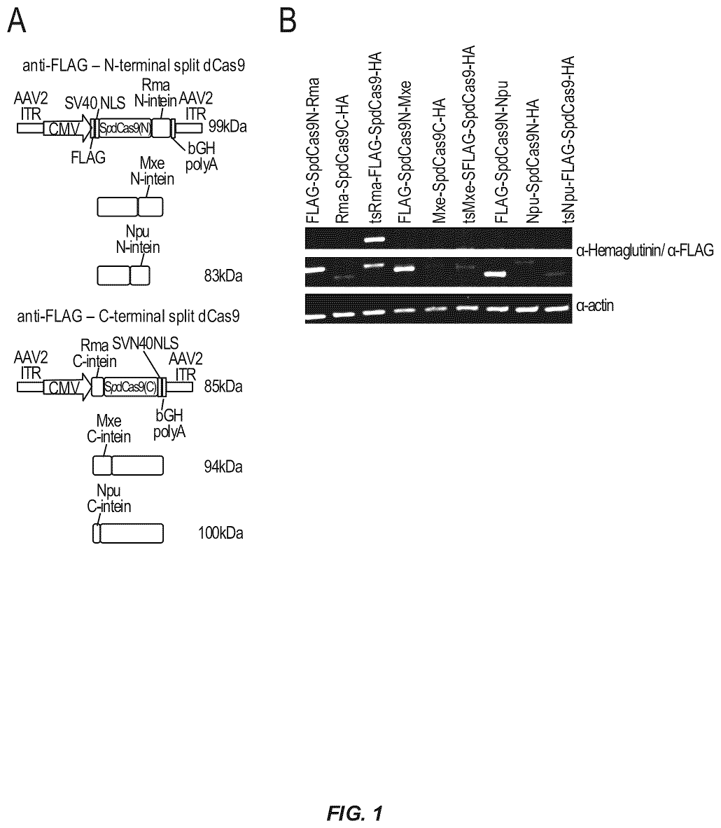

In order to test the feasibility of an intein-mediated split SpdCas9 approach that met the size restrictions of AAV packaging and to test delivery efficiency of a large SpdCas9 effector fusion protein into the central nervous system, several split SpdCas9 modalities were designed (FIG. 1A). Three bacteria-derived inteins were utilized to be fused to SpdCas9: Rhodothermus marinus (Rma) DnaB inteins (99 kDa N- and 85 kDa C-terminal split), Mycobacterium xenopi (Mxe) GyrA inteins (94 kDa per split), and the Nostoc punctiforme (Npu) DnaE inteins (83 kDa N and 100 kDa C-terminal split). Expression of the intein-fusion protein was driven by a small CMV promoter with a single SV40 NLS per split and a bGH polyA signal. The presence of a FLAG tag on the N-terminal split proteins and a C-terminal HA epitope allowed for detection of the expression of the individual proteins as well as trans-spliced full-length SpdCas9. In order to demonstrate reconstitution of SpdCas9, constructs were transfected individually or co-transfected into 293T cells. Transfection of the constructs resulted in the expression of all the individual N- and C-terminal intein-fusion proteins as demonstrated by Western Blot (FIG. 1B). Strikingly, the only full-length SpdCas9 protein that was detectable was mediated via RmaDnaB trans-splicing. This protein architecture configuration (tsdCas9) allows for the largest C-terminal effector protein and was used for subsequent proof-of-principle experiments.

In Vitro DNA Methylation Editing of CDKL5 Using Split Cas9

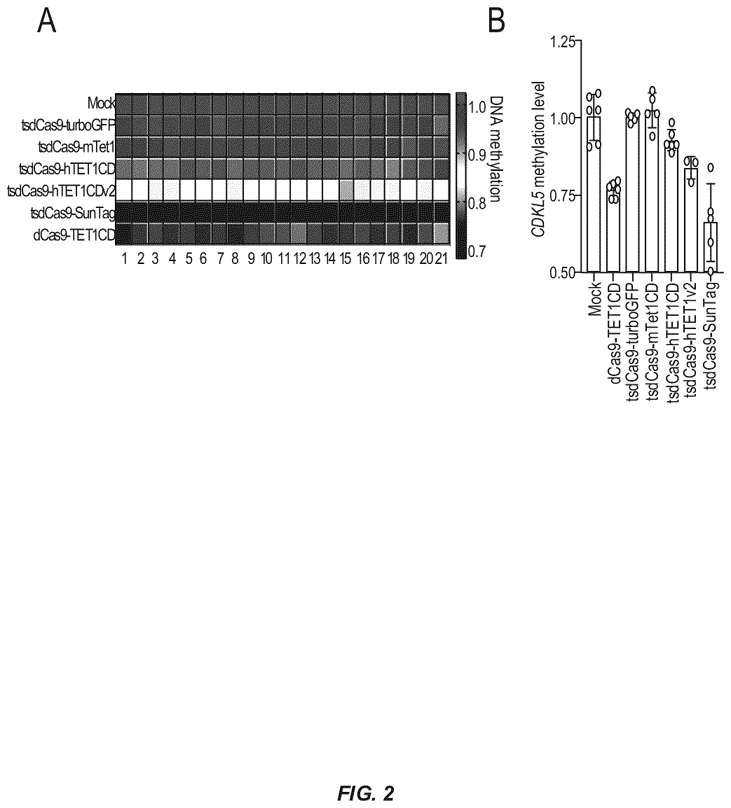

Due to DNA methylation editing of the CDKL5 promoter being a key mediator of gene reactivation, a split SpdCas9 platform is developed for targetable removal of DNA methylation (FIG. 2A). Several DNA demethylating protein domains were fused onto the C-terminal SpdCas9-RmaDnaB fusion protein, including the catalytic domain of human and the smaller murine Tet1 protein (hTET1CD and mTet1CD respectively). In addition, a TET1 protein with two additional nuclear localization signal (NLS) was generated to allow for efficient translocation of hTET1 into the nucleus. Furthermore, the establishment of a split three-piece SunTag peptide repeat array allowed for the recruitment of multiple hTET1CDs to the target site. Total DNA demethylation efficiency was assessed via targeted bisulfite amplicon sequencing of an amplicon spanning 21 CpG dinucleotides within the CDKL5 CGI promoter region in 293T cells co-transfected with the tsdCas9 construct and gRNAs targeting the CDKL5 promoter (FIG. 2B). Methylation levels of cells treated with a control tsdCas9-turboGFP fusion protein (arbitrarily set to 100% 5-meCG/CG±1.3%) were unchanged 72 h post-transfection when compared to mock-treated cells (100% 5-meCG/CG±7.5%, FIG. 2C). Cells transfected with a full-length SpdCas9-TET1CD positive control demonstrated a 23.6% decrease in DNA methylation when compared to mock-treated cells (p<0.0001). In addition, a mTet1 C-terminal fusion protein was unable to remove DNA methylation (100% 5-meCG/CG±1.3%) and no significant changes were observed upon delivery of hTET1CD (92.6%±3.4%). In contrast, the presence of additional NLS (tsdCas9-hTET1v2) resulted in a significant decrease of 16.6% 5-meCG/CG relative to mock-treated cells (p=0.012). Co-delivery of the three-piece tsdCas9SunTag resulted in a 34.2% decrease in DNA methylation across the assessed region (p=0.0041). Notably, there was a significant 17.6% decrease in cells treated with tsdCas9-SunTag when compared to tsdCas9-hTET1v2 (p=0.0091), indicating that the SunTag is the most efficient split DNA demethylating modality.

In Vitro Gene Regulation of CDKL5 Using tsdCas9 VP64 Demethylase Dual Fusions

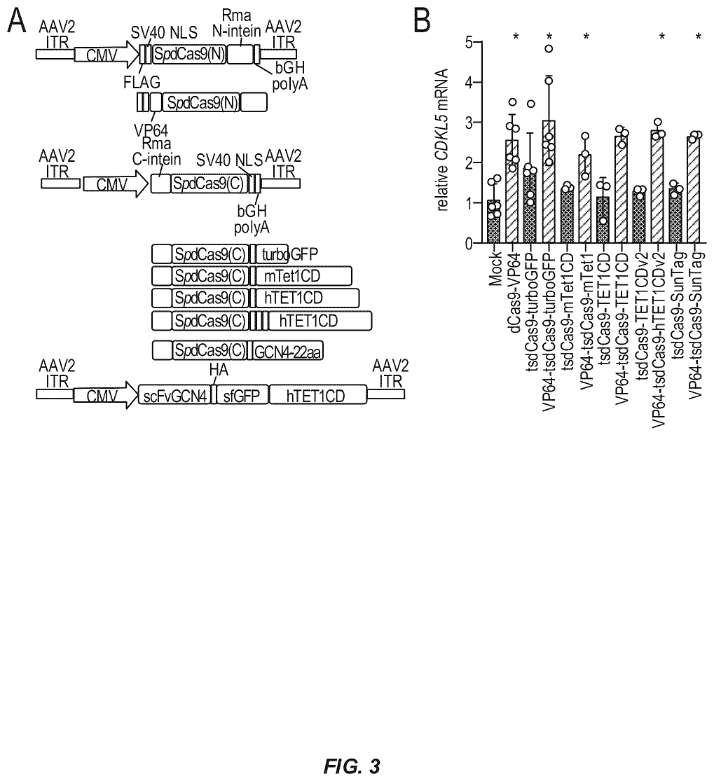

Next, it is determined whether the transcriptional activator VP64 fused to the N-terminal split dCas9 in combination with C-terminal SpdCas9 demethylase fusion proteins can be used for an all-in-one trans-splice system. In the past it was demonstrated that CDKL5 is amenable for programmable transcription via delivery of SpdCas9-VP64 across several cell lines. In order to determine efficiency of tsdCas9 dual effector fusions, constructs were co-transfected into 293T cells with gRNAs targeting the CDKL5 promoter (FIG. 3A). As demonstrated previously, a full-length SpdCas9-VP64 targeted to the gene promoter resulted in a significant 2.41-fold upregulation of CDKL5 when compared to mock-treated cells (p=0.0089) (FIG. 3B). Similarly, a 2.89-fold increase over mock was observed when using VP64-tsdCas9-turboGFP (p=0.0002). No significant difference was observed in the absence of a N-terminal VP64 fusion across the demethylase fusion proteins when compared to mock-treated cells. This demonstrates that the presence of a demethylase is not sufficient to upregulate total CDKL5 expression. In contrast, the addition of the demethylase effector domain resulted in significant gene upregulation for several constructs relative to mock, including VP64-tsdCas9-hTET1CD (2.52-fold, p=0.0336), VP64-tsdCas9-hTET1CDv2 (2.64-fold, p=0.0158) and VP64-tsdCas9-SunTag (2.51-fold, p=0.0357). CDKL5 gene expression was not significantly upregulated for the VP64-tsdCas9-mTet1CD fusion protein. The establishment of a dual effector fusion should further increase the likelihood of successful in vivo gene regulation.

Mild Reactivation of MECP2 Using a Trans-Spliced VP64 and TET1 Dual Effector Approach

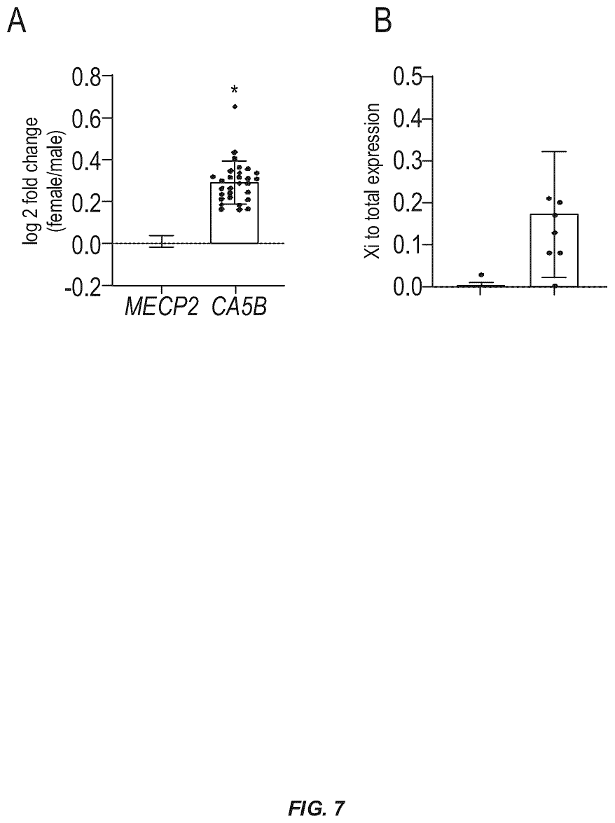

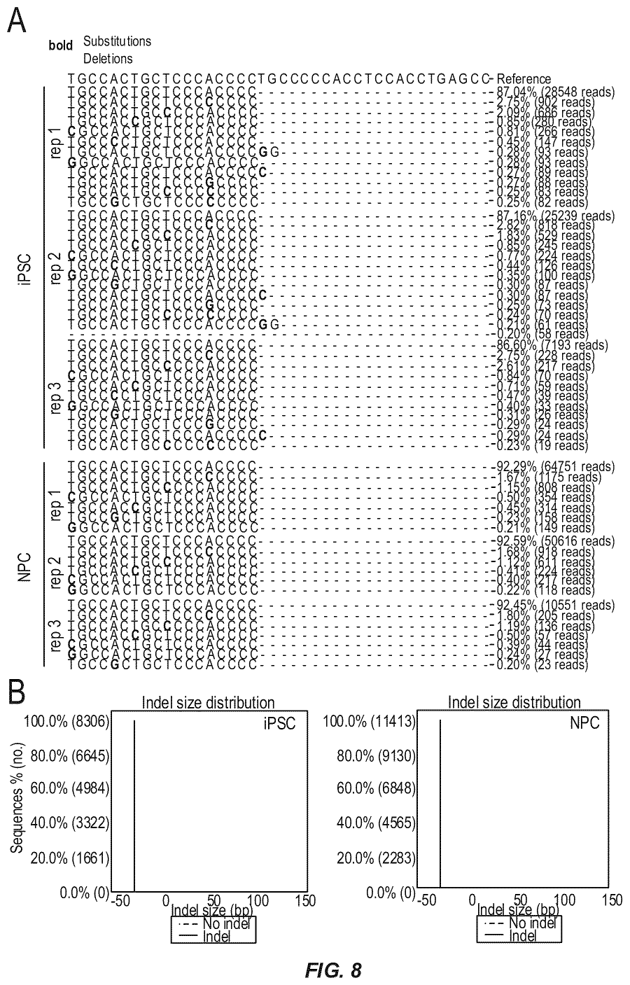

Two three-guide RNA combinations were then tested in a RTT NSC model with the novel trans-spliced(ts)dCas9 approach consisting of the VP64-split dCas9 and a SunTag peptide array. Previously reported data from a comparative analysis of bulk RNAseq data from GTeX across 29 different tissues(25) show that MECP2 does not display a bias in female over male expression ratio, which can be used as an indirect readout to determine the status of XCI status when compared to a known escape gene CA5B and hence would have higher expression in females than males (FIG. 7A). Furthermore, direct evidence from single cell RNA-seq indicates that MECP2 is not expressed from the inactive allele when compared to the same escape gene across multiple tissues (FIG. 1B). Taken together, these data indicate that MECP2 is not an escapee. This was confirmed by deep sequencing that the RTT iPSC line is clonal for the 32 bp deletion in MECP2, expressing only the mutant allele (FIG. 8). Differentiation of iPSCs in NSCs revealed the same clonality pattern. Therefore, downstream reactivation analysis should not be confounded by mosaic expression of the mutant or intact allele.

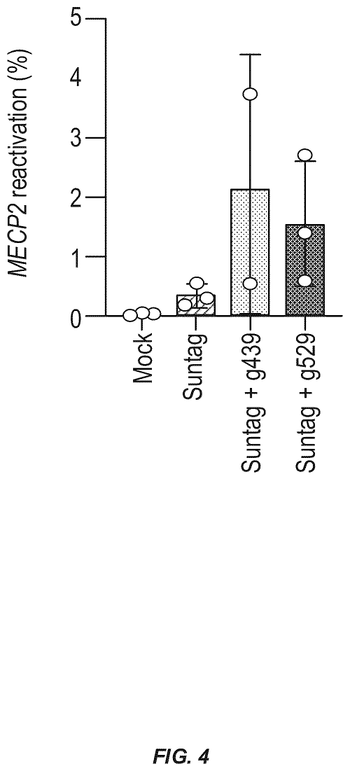

NSCs were co-transfected using Lipofectamine Stem reagent with the tsdCas9-SunTag and the two lead guide RNA combinations. Seventy-two hours following transfection, amplicon sequencing was performed to detect the level of wild-type MECP2 reactivation. With the two lead guide RNA combinations g5g1g7 and g6g2g8 reactivation of the silent, wild-type MECP2 was observed (FIG. 4). Mock-treated cells demonstrated less than 0.03% expression of the wild-type allele. Overexpression of the split tsdCas9-SunTag system resulted in a ten-fold increase of mRNA expression from the inactive allele (0.34%), likely due to global DNA hypomethylation. Lastly, targeting of the promoter with the two lead guide RNA combinations previously demonstrated to remove DNA methylation resulted in a 2.1% and 1.5% expression from the inactive allele for g4g3g9 and g5g2g9, respectively.

In Vivo Distribution and Reconstitution of Split SpdCas9 Via AAV9

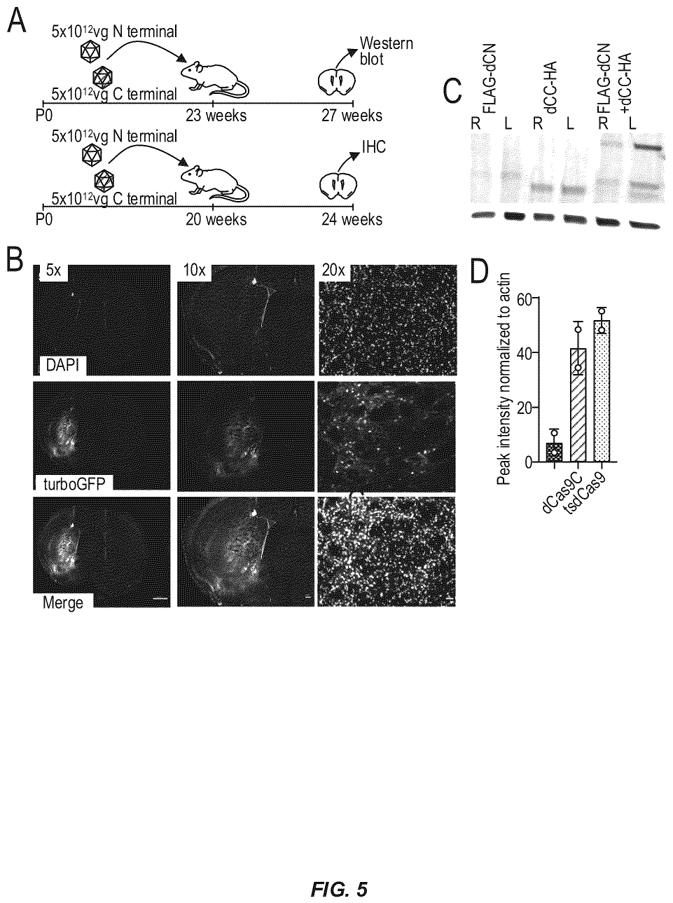

In order to demonstrate expression and transduction throughout the mouse brain of AAV9-mediated SpdCas9 delivery and in vivo reconstitution of split dCas9 proteins, stereotaxic injections into the striatum were performed in 23-week old wild-type FVB mice (FIG. 5A). Animals were either unilaterally injected with a C-terminal dCas9-P2A-turboGFP for distribution or bilaterally injected with N-terminal FLAG- and C-terminal HA-tagged split SpdCas9 particles, or a combination thereof for full-length trans-splicing. Twenty-one days post-treatment robust expression of dCas9C-turboGFP and widespread AAV9 transduction as demonstrated by the expression of turboGFP in the striatum was observed (FIG. 5B). In addition, the presence of N- and C-terminal dCas9 split proteins was detected in animals that were bilaterally injected with AAV9 (FIG. 4C). Finally, animals that were co-transduced with AAV9 demonstrated robust in vivo reconstitution of tsdCas9, with more than 50% of total protein in the trans-spliced dCas9 configuration (FIG. 4D). These data demonstrate efficient trans-splicing of the tsdCas9 approach for in vivo gene regulation.

Simultaneous gRNA Expression from a Single tRNA-gRNA Cassette

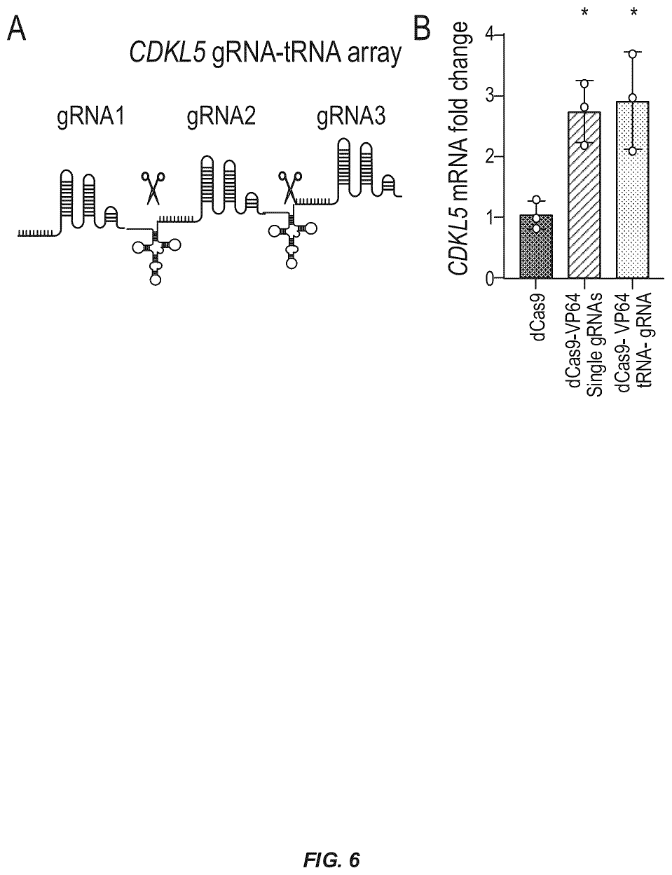

In order to further overcome the packaging size of the AAV backbone, the number of U6 promoters was reduced for the expression of the three CDKL5 lead gRNAs via the introduction of a gRNA-tRNA array. In this approach, the U6 promoter drives the expression of a single gRNA that is interspersed by cleavable glycine tRNAs that result in the expression of individual gRNAs (FIG. 6A). In order to demonstrate programmable transcription of the CDKL5 gene using a multiplex system, 293T cells were co-transfected with a full-length dCas9 and dCas9-VP64 with individual gRNAs as well as a SpdCas9-VP64 with the tRNA-gRNA assay (FIG. 6B). Targeting the CDKL5 promoter with individual gRNAs resulted in a significant 2.7-fold increase when compared to a dCas9 without effector domains (p=0.0237). Similarly, when CDKL5 multiplex targeting was performed using the tRNA-gRNA cassette a significant 2.9-fold change was observed relative to SpdCas9 alone (p=0.0153). This approach eliminates the necessity for multiple U6 sgRNA cassettes.

Discussion

The present inventors' research group has previously demonstrated that epigenetic reactivation of the CDKL5 gene on the X-chromosome is achievable via lentiviral co-transduction of two CRISPR/dCas9 epigenetic editor fusion proteins for simultaneous DNA demethylation and gene upregulation(20). In order to make the epigenetic editing platform feasible for the clinic, the hurdle of delivery of a biological into the central nervous system needs to be cleared and carefully vetted using in vivo proof of concept studies. In this study, the inventors demonstrate that a dual split dCas9 effector approach linked with a gRNA-tRNA array for CDKL5 gRNA multiplexing is functional in vitro and AAV deliverable into the brain of wildtype mice, allowing for future functional studies in vivo. Indeed, promising work by others demonstrates that epigenetic rescue of X-linked disease is possible(29) and that delivery of VP64 via a S. aureus dCas9 fusion protein has proven efficacious in the treatment of a haploinsufficiency(30). It is important to note that, as of now no DNA demethylating strategy utilizing an AAV-ready backbone has been utilized. Here, the present inventors demonstrate that efficient hTET1 demethylase is likely dependent on translocation of the protein to the nucleus, since the addition of two extra NLS tags were necessary for removal of DNA methylation. In addition, a smaller murine Ted protein was unable to edit DNA methylation, which could be caused by inefficient nuclear localization. Future studies will need to evaluate expression of the construct by Western Blot. Furthermore, it is observed that an AAV-ready tsdCas9-SunTag demonstrates the highest DNA demethylation across the constructs tested(31). In addition, preliminary data indicates that deliver of tsdCas9-SunTag results in mild reactivation of MECP2 in patient-derived cells.

In this study, three previously reported inteins were tested. Truong et al. constructed two versions of trans-spliced SpCas9, either split between Glu573 and Cys574 or Lys637 and Thr638, using Npu DnaE inteins for the generation of CRISPR mediated non homologous end joining across several targets in mouse and human cell lines. In addition, Truong et al. demonstrated that an intein-mediated SpCas9 D10A nickase was functional, albeit to a lower degree than full-length SpCas9 D10A, and due to size reduction allowed for the incorporation of donor DNA into the same AAV for homology directed repair(17). While in this study the Cys574 split did not result in trans-splicing, this does not rule out the possibility that the other breakpoint in the SpdCas9 protein will result in efficient trans-splicing. However, this alternative site would limit the packaging capability on the C-terminal split dCas9. Similar to the previous study, the Bao lab demonstrated that nuclease activity in human cells was attainable at about one-third of the activity level of wild-type SpCas9 using Mxe-derived GyrA inteins. Since the GyrA intein depends on a YT motif found in the SpCas9 polypeptide sequence, only a split at Y656 resulted in a meaningful size reduction of N- and C-terminal SpCas9. Strikingly, SpCas9 reconstitution was not equally efficient in every cell line transfected, since K562 and HeLa cells failed to undergo trans-splicing for unknown reasons (19). This study recapitulated these findings in HEK293T cells, not ruling out the possibility that the Mxe GyrA inteins are not able to undergo trans-splicing in different more clinically relevant cell lines. It is important to note, however, that instead of a reduction in activity being observed, DNA demethylation and gene upregulation appeared to be as effective as published full-length SpdCas9 constructs.

In this study, the Rma intein system was the only split dCas9 that was able to trans-splice in vitro and in vivo and elicit functional effects in vitro, including in patient-derived cells. Previous data suggested that intraperitoneal delivery as well as intramuscular DNA electroporation of Rma-tsCas9 was able to edit genes in neonatal mice. Of interest, target engagement as well as off-target effects positively correlated with the viral genomes per cell. With regards to the current work, the split system allowed for viral delivery of a C-terminal half of a VPR transcription activator fused to a SpCas9 nuclease, which demonstrated modest targeted upregulation of endogenous genes in vivo. Importantly, a cellular and humoral immune response was observed in mice, regardless of how SpCas9 was delivered. Strikingly, when changing the residues that were found to be enriched by epitope mapping in the immune response, SpCas9 protein was still retaining its function(32). This holds great promise for reducing the potential immunogenicity of Cas9 therapeutics in future studies.

From a preclinical perspective, the potential of a split Cas9 delivered by AAV was further highlighted by several publications targeting different disease indications. This includes SpdCas9 mediated epigenetic gene repression in vivo via tail vein injection of AAV8 using a bipartite effector protein configuration(33). In this study, constructs were generated that are not dependent on bipartite expression of the effectors, but rather dependent on the expression of VP64 and TET1 from either termini, further increasing packaging capacity per split protein. Subretinal injection of a split dCas9 KRAB repressor fusion into a mouse model of autosomal recessive retinitis pigmentosa at P7 demonstrated prevention of photoreceptor degeneration and largely improved visual acuity(33). Additionally, different strategies have been adapted for in vivo SpCas9 mediated cytidine and adenine base editing. Intravenous injection of base editors using AAV8 in a model of phenylketonuria demonstrated long-term DNA correction and efficacy over a 28-week time span post-delivery(34). Similarly, base editing has been utilized for exon-skipping in the X-linked disorder Duchenne's muscular dystrophy via direct targeting of skeletal muscle cells in a pig model of the disease via AAV9 intein-split Cas9(35). The Perez-Pinera group further refined the cytidine base editing strategy and applied it to a heritable form of amyotrophic lateral sclerosis caused by mutations in the SOD1 gene(36). Split-intein CRISPR base editor increased survival in a mouse model of the disease, reducing muscular atrophy and denervation as well as improving overall neuromuscular function and fewer immunoreactive inclusions in motor neurons. Finally, the Liu et al. group also utilized base editors using the Cys574 Npu intein delivered by retro-orbital injection of AAV9(37). They detected adenine and cytosine base editing in multiple organs, with the highest transduction and concomitantly editing rate in the liver. Strikingly, high editing efficiency was described in the cortex and cerebellum of C57BL/6 mice at P0 and at 9 weeks of age. When applied to a mouse model of the neurodegenerative Niemann-Pick disease, targeting Npc1, lifespan in these mice was significantly increased, concomitantly with an increase in Purkinje cell survival and reduced inflammation. Importantly, their approach resulted in high transduction of the brain, including more than 50% of cortical neurons. Taken together, these studies highlight the possibility of non-invasive AAV delivery routes and may be adaptable for the central nervous system. This study demonstrates that robust transduction and trans-splicing occur for more than 50% of the total protein content.

In addition to alterations in the protein configuration, additional studies will be required to evaluate the potential of ribozyme splicing systems as an alternative to gRNA-tRNA arrays for multiplexed gRNA expression from a single promoter(38), as well as address promoter orientation questions and the addition of post-transcriptional regulatory elements that may increase transgene expression(37).

While mouse models provide an excellent tool to study disease etiology and neurological phenotypes on the behavioral and motor skill level, it is important to keep in mind that novel customizable epigenetic modifiers require the necessity to target the human genome. One compelling tool to study such epigenetic editors is represented by the utility of patient-derived iPSC. This study demonstrates that patient derived cells from RTT can be clonally propagated and allow the assessment of wild-type allele reactivation. Due to the loss of the confounding mosaicism, X-reactivation approaches in such models can be rapidly used for the testing of rescue of neuronal phenotypes, such as aberrant neuronal firing, dendritic arborization and pre- and post-synaptic impairments on the molecular level. Importantly, the amount of reactivation observed in the RTT NSC line is comparable to previously published data. The Lee lab demonstrated that a mixed modality approach using an ASO targeted against XIST and small molecule inhibition of DNA methylation resulted in 3% reactivation of MECP2. Noteworthy, brain-wide ablation of Xist in a Nestin-driven transgenic mouse model of RTT did not result in decreased life-span relative to wild-type animals, indicating that post-mitotic ablation is well tolerated. In addition, co-treatment of 5-aza in neuronal specific Xist deletion transgenic animals resulted in an overall cumulative increase in expression of genes stemming from the X-chromosome, highlighting the potential detrimental impact that global X-reactivation may have on the organism(39). While promising for disorders of the CNS, selective small molecule inhibition of XIST that is specific to the brain remains elusive. Since previous data indicate that loss of XIST resulted in hematological cancer in mice(40), as would be the result of systemic delivery, the approach developed in this study has the advantage of being selective for the disease causative gene.

Future studies will evaluate the impact that further expansion has on RTT iPSC and NSC clonality. Several studies suggest that X-chromosome inactivation can be erosive in iPSCs. Previous data demonstrate that non-random X-chromosome reactivation in regions that are particularly enriched for H3K27me3 can occur(41). This process is likely mediated via the expression of a long-non coding RNA, termed XACT from the X-inactivation center(42). While this phenomenon was not observed in the current study, this highlights the importance of having carefully controlled experiments in the future that will evaluate baseline reactivation levels in such patient-derived cells.

Taken together, gene reactivation strategies are of high interest to the field of X-linked neurological disorders. These predominantly affect females due to dominant gene mutations, leaving a druggable, silenced, wildtype allele behind that potentially can rescue phenotypes when reactivated using a tsdCas9 epigenetic editing approach. While still in its infancy, in vivo epigenetic editing, as demonstrated by us and others, has the potential to become essential for novel gene therapies.

All patents, patent applications, and other publications, including GenBank Accession Numbers and equivalents, cited in this application are incorporated by reference in the entirety for all purposes.

### REFERENCES

1. Li, C. and Samulski, R. J. (2020) Engineering adeno-associated virus vectors for gene therapy. Nat. Rev. Genet., 10.1038/s41576-019-0205-4.
2. Wu, Z., Yang, H. and Colosi, P. (2010) Effect of genome size on AAV vector packaging. Mol. Ther., 18, 80-86.
3. Jinek, M., Chylinski, K., Fonfara, I., Hauer, M., Doudna, J. A. and Charpentier, E. (2012) A programmable dual-RNA-guided DNA endonuclease in adaptive bacterial immunity. Science, 337, 816-821.
4. Cong, L., Ran, F. A., Cox, D., Lin, S., Barretto, R., Habib, N., Hsu, P. D., Wu, X., Jiang, W., Marraffini, L. A., et al. (2013) Multiplex genome engineering using CRISPR/Cas systems. Science, 339, 819-823.
5. Mali, P., Yang, L., Esvelt, K. M., Aach, J., Guell, M., DiCarlo, J. E., Norville, J. E. and Church, G. M. (2013) RNA-guided human genome engineering via Cas9. Science, 339, 823-826.
6. Cho, S. W., Kim, S., Kim, J. M. and Kim, J.-S. (2013) Targeted genome engineering in human cells with the Cas9 RNA-guided endonuclease. Nat. Biotechnol., 31, 230-232.
7. Miyaoka, Y., Chan, A. H., Judge, L. M., Yoo, J., Huang, M., Nguyen, T. D., Lizarraga, P. P., So, P.-L. and Conklin, B. R. (2014) Isolation of single-base genome-edited human iPS cells without antibiotic selection. Nat. Methods, 11, 291-293.
8. Mali, P., Aach, J., Stranges, P. B., Esvelt, K. M., Moosburner, M., Kosuri, S., Yang, L. and Church, G. M. (2013) CAS9 transcriptional activators for target specificity screening and paired nickases for cooperative genome engineering. Nat. Biotechnol., 31, 833-838.
9. Gaudelli, N. M., Komor, A. C., Rees, H. A., Packer, M. S., Badran, A. H., Bryson, D. I. and Liu, D. R. (2017) Programmable base editing of A T to G C in genomic DNA without DNA cleavage. Nature, 551, 464-471.
10. Maeder, M. L., Linder, S. J., Cascio, V. M., Fu, Y., Ho, Q. H. and Joung, J. K. (2013) CRISPR RNA-guided activation of endogenous human genes. Nat. Methods, 10, 977-979.
11. Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013) RNA-guided gene activation by CRISPR-Cas9-based transcription factors. Nat. Methods, 10, 973-976.
12. Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014) Genome-Scale CRISPR-Mediated Control of Gene Repression and Activation. Cell, 159, 647-661.
13. Thakore, P. I., Black, J. B., Hilton, I. B. and Gersbach, C. A. (2016) Editing the epigenome: technologies for programmable transcription and epigenetic modulation. Nat. Methods, 13, 127-137.
14. Nishimasu, H., Ran, F. A., Hsu, P. D., Konermann, S., Shehata, S. I., Dohmae, N., Ishitani, R., Zhang, F. and Nureki, O. (2014) Crystal structure of Cas9 in complex with guide RNA and target DNA. Cell, 156, 935-949.
15. Wright, A. V, Sternberg, S. H., Taylor, D. W., Staahl, B. T., Bardales, J. A., Kornfeld, J. E. and Doudna, J. A. (2015) Rational design of a split-Cas9 enzyme complex. Proc. Natl. Acad. Sci. U.S.A, 112, 2984-2989.
16. Shah, N. H. and Muir, T. W. (2014) Inteins: nature's gift to protein chemists. Chem. Sci., 5, 446-461.
17. Truong, D.-J. J., Werfel, S., Engelhardt, S., Wurst, W. and Ortiz, O. (2015) Development of an intein-mediated split-Cas9 system for gene therapy. Nucleic Acids Res., 43, 6450-6458.
18. Chew, W. L., Tabebordbar, M., Cheng, J. K. W., Mali, P., Wu, E. Y., Ng, A. H. M., Zhu, K., Wagers, A. J. and Church, G. M. (2016) A multifunctional AAV—CRISPR-Cas9 and its host response. Nat. Methods, 13, 868-874.
19. Fine, E. J., Appleton, C. M., White, D. E., Brown, M. T., Deshmukh, H., Kemp, M. L. and Bao, G. (2015) Trans-spliced Cas9 allows cleavage of HBB and CCRS genes in human cells using compact expression cassettes. Sci. Rep., 5, 10777.
20. Halmai, J. A. N. M., Deng, P., Gonzalez, C. E., Coggins, N. B., Cameron, D., Carter, J. L., Buchanan, F. K. B., Waldo, J. J., Lock, S. R., Anderson, J. D., et al. (2020) Artificial escape from XCI by DNA methylation editing of the CDKL5 gene. Nucleic Acids Res., 10.1093/nar/gkz1214.
21. Kabadi, A. M., Ousterout, D. G., Hilton, I. B. and Gersbach, C. A. (2014) Multiplex CRISPR/Cas9-based genome engineering from a single lentiviral vector. Nucleic Acids Res., 42, e147.
22. Zhang, Y., Wang, J., Wang, Z., Zhang, Y., Shi, S., Nielsen, J. and Liu, Z. (2019) A gRNA-tRNA array for CRISPR-Cas9 based rapid multiplexed genome editing in Saccharomyces cerevisiae. Nat. Commun., 10, 1-10.
23. Kalscheuer, V. M., Tao, J., Donnelly, A., Hollway, G., Schwinger, E., Kubart, S., Menzel, C., Hoeltzenbein, M., Tommerup, N., Eyre, H., et al. (2003) Disruption of the serine/threonine kinase 9 gene causes severe X-linked infantile spasms and mental retardation. Am. J. Hum. Genet., 72, 1401-1411.
24. Amir, R. E., Van den Veyver, I. B., Wan, M., Tran, C. Q., Francke, U. and Zoghbi, H. Y. (1999) Rett syndrome is caused by mutations in X-linked MECP2, encoding methyl-CpG-binding protein 2. Nat. Genet., 23, 185-188.
25. Tukiainen, T., Villani, A.-C., Yen, A., Rivas, M. A., Marshall, J. L., Satija, R., Aguirre, M., Gauthier, L., Fleharty, M., Kirby, A., et al. (2017) Landscape of X chromosome inactivation across human tissues. Nature, 550, 244.
26. Li, L.-C. and Dahiya, R. (2002) MethPrimer: designing primers for methylation PCRs. Bioinformatics, 18, 1427-1431.
27. Krueger, F. and Andrews, S. R. (2011) Bismark: a flexible aligner and methylation caller for Bisulfite-Seq applications. Bioinformatics, 27, 1571-1572.
28. Guo, W., Zhu, P., Pellegrini, M., Zhang, M. Q., Wang, X. and Ni, Z. (2018) CGmapTools improves the precision of heterozygous SNV calls and supports allele-specific methylation detection and visualization in bisulfite-sequencing data. Bioinformatics, 34, 381-387.
29. Liu, X. S., Wu, H., Krzisch, M., Wu, X., Graef, J., Muffat, J., Hnisz, D., Li, C. H., Yuan, B., Xu, C., et al. (2018) Rescue of Fragile X Syndrome Neurons by DNA Methylation Editing of the FMR1 Gene. Cell, 172, 979-992.e6.
30. Matharu, N., Rattanasopha, S., Tamura, S., Maliskova, L., Wang, Y., Bernard, A., Hardin, A., Eckalbar, W. L., Vaisse, C. and Ahituv, N. (2019) CRISPR-mediated activation of a promoter or enhancer rescues obesity caused by haploinsufficiency. Science (80-.), 363.
31. Morita, S., Noguchi, H., Horii, T., Nakabayashi, K., Kimura, M., Okamura, K., Sakai, A., Nakashima, H., Hata, K. and Nakashima, K. (2016) Targeted DNA demethylation in vivo using dCas9-peptide repeat and scFv—TET1 catalytic domain fusions. Nat. Biotechnol., 34, 1060-1065.
32. Chavez, A., Scheiman, J., Vora, S., Pruitt, B. W., Tuttle, M., P R Iyer, E., Lin, S., Kiani, S., Guzman, C. D., Wiegand, D. J., et al. (2015) Highly efficient Cas9-mediated transcriptional programming. Nat. Methods, 12, 326-328.
33. Moreno, A. M., Fu, X., Zhu, J., Katrekar, D., Shih, Y.-R. V, Marlett, J., Cabotaje, J., Tat, J., Naughton, J., Lisowski, L., et al. (2018) In Situ Gene Therapy via AAV—CRISPR-Cas9-Mediated Targeted Gene Regulation. Mol. Ther., 26, 1818-1827.
34. Villiger, L., Grisch-Chan, H. M., Lindsay, H., Ringnalda, F., Pogliano, C. B., Allegri, G., Fingerhut, R., Haberle, J., Matos, J., Robinson, M. D., et al. (2018) Treatment of a metabolic liver disease by in vivo genome base editing in adult mice. Nat. Med., 24, 1519-1525.
35. Winter, J., Luu, A., Gapinske, M., Manandhar, S., Shirguppe, S., Woods, W. S., Song, J. S. and Perez-Pinera, P. (2019) Targeted exon skipping with AAV-mediated split adenine base editors. Cell Discov., 5, 41.
36. Lim, C. K. W., Gapinske, M., Brooks, A. K., Woods, W. S., Powell, J. E., Zeballos C, M. A., Winter, J., Perez-Pinera, P. and Gaj, T. (2020) Treatment of a Mouse Model of ALS by In Vivo Base Editing. Mol. Ther., 10.1016/j.ymthe.2020.01.005.
37. Levy, J. M., Yeh, W.-H., Pendse, N., Davis, J. R., Hennessey, E., Butcher, R., Koblan, L. W., Comander, J., Liu, Q. and Liu, D. R. (2020) Cytosine and adenine base editing of the brain, liver, retina, heart and skeletal muscle of mice via adeno-associated viruses. Nat. Biomed. Eng., 4, 97-110.
38. Xu, L., Zhao, L., Gao, Y., Xu, J. and Han, R. (2017) Empower multiplex cell and tissue-specific CRISPR-mediated gene manipulation with self-cleaving ribozymes and tRNA. Nucleic Acids Res., 45, e28-e28.
39. Carrette, L. L. G., Wang, C.-Y., Wei, C., Press, W., Ma, W., Kelleher, R. J. and Lee, J. T. (2018) A mixed modality approach towards Xi reactivation for Rett syndrome and other X-linked disorders. Proc. Natl. Acad. Sci., 115, E668 LP-E675.
40. Yildirim, E., Kirby, J. E., Brown, D. E., Mercier, F. E., Sadreyev, R. I., Scadden, D. T. and Lee, J. T. (2013) Xist RNA is a potent suppressor of hematologic cancer in mice. Cell, 152, 727-742.
41. Vallot, C., Ouimette, J.-F., Makhlouf, M., Féraud, O., Pontis, J., Come, J., Martinat, C., Bennaceur-Griscelli, A., Lalande, M. and Rougeulle, C. (2015) Erosion of X Chromosome Inactivation in Human Pluripotent Cells Initiates with XACT Coating and Depends on a Specific Heterochromatin Landscape. Cell Stem Cell, 16, 533-546.
42. Vallot, C., Patrat, C., Collier, A. J., Huret, C., Casanova, M., Liyakat Ali, T. M., Tosolini, M., Frydman, N., Heard, E., Rugg-Gunn, P. J., et al. (2017) XACT Noncoding RNA Competes with XIST in the Control of X Chromosome Activity during Human Early Development. Cell Stem Cell, 20, 102-111.

### VII. Exemplary Embodiments

Exemplary embodiments provided in accordance with the herein disclosed subject matter include, but are not limited to, the claims and following embodiments:

1. An epigenetic editing system comprising: (i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an epigenetic modifier.

2. The system of embodiment 1, further comprising a third expression cassette comprising a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site.

3. The system of embodiment 1, wherein the first and/or second expression cassettes further comprise a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of the target gene transcription start site.

4. The system of any one of embodiments 1-3, wherein the transcription activator is VP64, an MS2-loop SAM system, a mini-VPR, p30000RE, or any combination thereof.

5. The system of any one of embodiments 1-4, wherein the dCas9 protein is a Streptococcus pyogenes dCas9 (spdCas9) protein.

6. The system of embodiment 5, wherein the N-dCas9 and C-dCas9 consist of the 1 to 713 segment and the 713 to 1368 segment of SEQ ID NO:1, respectively.

7. The system of any one of embodiments 1-6, wherein the intein is Rhodothermus marinus (Rma) DNA helicase DnaB.

8. The system of embodiment 7, wherein the N-intein and C-intein consist of the 1 to 102 segment and the 103 to 154 segment of SEQ ID NO:2, respectively.

9. The system of any one of embodiments 1-8, wherein the epigenetic modifier is a human Ten-Eleven Translocation methylcytosine dioxygenase 1 catalytic domain (hTET1CD), a Suntag, a DOT1L catalytic domain, PRDM9CD, an amoeba Tet1 (NgTet1), or any combination thereof.

10. The system of any one of embodiments 1-9, wherein each of the first, second, or third polynucleotide sequence is operably linked to a promoter and optionally further to a polyA sequence.

11. The system of embodiment 10, wherein the promoter is a CMV promoter.

12. The system of any one of embodiments 1-11, wherein the N-terminal split protein further comprises at least one nuclear localization signal (NLS) located at the N-terminus to the transcription activator.

13. The system of any one of embodiments 1-12, wherein the C-terminal split protein further comprises at least one NLS, preferably two or three NLS, located between the C-dCas9 and the epigenetic modifier.

14. The system of embodiment 12 or 13, wherein the NLS is an SV40 NLS.

15. The system of any one of embodiments 1-14, wherein the first, second, or third expression cassette comprises a coding sequence encoding two or three sgRNAs.

16. The system of any one of embodiments 2-15, wherein the first, second, and third expression cassettes are present in three separate vectors.

17. The system of embodiment 16, wherein each of the vectors is a viral vector or a plasmid.

18. The system of embodiment 17, wherein the viral vector is a lentiviral vector, an adeno-associated viral (AAV) vector, or an adenoviral vector.

19. The system of any one of embodiments 2-18, wherein the target gene is CDKL5.

20. The system of any one of embodiments 2-19, wherein the target sequence comprises or consists of AGAGCATCGGACCGAAGCGG (SEQ ID NO:12), GGGGGAGAACATACTCGGGG (SEQ ID NO:13), or CCCAGGTTGCTAGGGCTTGG (SEQ ID NO:14).

21. A host cell comprising the system of any one of embodiments 1-20.

22. The host cell of embodiment 21, which is a mammalian cell.

23. The host cell of embodiment 21 or 22, which is a human cell.

24. The host cell of any one of embodiments 21-23, which is an induced pluripotent stem cell (iPSC) or a neural stem cell (NSC).

25. A host cell comprising (i) an N-terminal split protein, which comprises, from its N-terminus, a transcription activator, N-terminal half of a catalytically inactive Cas9 protein (N-dCas9), and N-terminal half of an intein (N-intein); (ii) a C-terminal split protein, which comprises, from its N-terminus, C-terminal half of the intein (C-intein), C-terminal half of the dCas9 protein (C-dCas9), and an epigenetic modifier; and (iii) at least one small guide RNA (sgRNA), each of which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site.

26. The host cell of embodiment 25, wherein the N-terminal split protein further comprises at least one NLS located at the N-terminus to the transcription activator, and/or wherein the C-terminal split protein further comprises at least one NLS, preferably two or three NLS, located between the C-dCas9 and the epigenetic modifier.

27. A host cell comprising (i) a fusion protein, which comprises, from its N-terminus, a transcription activator, N-dCas9, C-dCas9, and an epigenetic modifier; and (ii) at least one small guide RNA (sgRNA), each of which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site.

28. The host cell of embodiment 27, wherein the fusion protein further comprises at least one NLS located at the N-terminus to the transcription activator, the at least one NLS, preferably two or three NLS, located between the C-dCas9 and the epigenetic modifier.

29. The host cell of embodiment 26 or 28, wherein the NLS is an SV40 NLS.

30. The host cell of any one of embodiments 21-29, wherein the target gene is CDKL5.

31. A composition comprising the system of any one of embodiments 1-20 or the host cell of any one of embodiments 21-30, optionally with a pharmaceutically acceptable carrier.

32. A method for modulating a target gene expression in a cell, comprising introducing into the cell an effective amount of a composition comprising the system of any one of embodiments 1-20, thereby modulating the target gene expression.

33. The method of embodiment 32, wherein the cell is a mammalian cell.

34. The method of embodiment 32 or 33, wherein the cell is a human cell.

35. The method of any one of embodiments 32-34, wherein the cell is a neuronal cell.

36. The method of any one of embodiments 32-34, wherein the cell is an induced pluripotent stem cell (iPSC) or a neural stem cell (NSC).

37. The method of any one of embodiments 32-36, wherein the target gene is CDKL5, wherein the method is for increasing CDKL5 gene expression in a cell with a hypermethylated CDKL5 promoter and suppressed CDKL5 expression, and wherein introducing into the cell the effective amount of the composition comprising the system of any one of embodiments 1-20 increases CDKL5 gene expression.

38. A method for treating CDKL5 deficiency disorder (CDD) in a subject in need thereof, comprising administering to the subject an effective amount of each of: (i) a first expression cassette comprising a first polynucleotide sequence encoding an N-terminal split protein, which comprises, from its N-terminus, a transcription activator, N-terminal half of a catalytically inactive Cas9 protein (N-dCas9), and N-terminal half of an intein (N-intein); and (ii) a second expression cassette comprising a second polynucleotide sequence encoding a C-terminal split proteion, which comprises, from its N-terminus, C-terminal half of the intein (C-intein), C-terminal half of the dCas9 protein (C-dCas9), and an epigenetic modifier, thereby increasing CDKL5 gene expression in the subject.

39. The method of embodiment 38, further comprising administering to the subject an effective amount of (iii) a third expression cassette comprising a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of CDKL5 gene transcription start site. CDKL5 gene expression in the subject.

40. The method of embodiment 38, wherein the first and/or second expression cassette further comprises a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of CDKL5 gene transcription start site.

41. The method of any one of embodiments 38-40, wherein the first, second, and/or third expression cassettes are administered to the subject in one composition.

42. The method of any one of embodiments 38-40, wherein the first, second, and/or third expression cassettes are administered to the subject in two or three compositions.

43. The method of any one of embodiments 38-42, wherein the subject is an infant or a juvenile human.

44. The method of any one of embodiments 38-42, wherein the subject is an adult human.

45. A method for treating CDKL5 deficiency disorder (CDD) in a subject in need thereof, comprising administering to the subject an effective amount of the host cell of any one of embodiments 21-30.

46. The method of embodiment 45, wherein the host cell is an induced pluripotent stem cell (iPSC) or a neural stem cell (NSC).

47. The method of any one of embodiments 38-46, wherein the administering step comprises intravenous, intranasal, intracranial, intrathecal, or intracisternal magna administration.

TABLE 1
Spacer Sequences Used to Create Target-Specific sgRNA Expression Vectors

| Oligonucleotide Name | Function | 5′->3′ Sequence |
|---|---|---|
| CDKL5 sgRNA1 | spacer sequence | AGAGCATCGGACCGAAGCGG (SEQ |
| | | ID NO: 18) |
| CDKL5 sgRNA2 | spacer sequence | GGGGGAGAACATACTCGGGG (SEQ |
| | | ID NO: 19) |
| CDKL5 sgRNA3 | spacer sequence | CCCAGGTTGCTAGGGCTTGG (SEQ |
| | | ID NO: 20) |
| CDKL5 sgRNA4 | spacer sequence | ATCGCCTGAA ACTTGTCCGG (SEQ |
| | | ID NO: 21) |
| CDKL5 sgRNA5 | spacer sequence | CGAAAGGGTGTGAAAGAGGG (SEQ |
| | | ID NO: 22) |
| CDKL5 sgRNA6 | spacer sequence | TGGGGAAGGTAAAGCGGCGA (SEQ |
| | | ID NO: 23) |
| rs1808_gDNA_F | Sanger sequencing | GCTTGAGCAATTTCGGACCC (SEQ |
| | | ID NO: 24) |
| rs1808_gDNA_R | Sanger sequencing | TGTGTCTCTTGCTGGTACCG (SEQ |
| | | ID NO: 25) |
| rs35478150_gDNA_F | Sanger sequencing | TGAGCCTGTGCCAGAGGATA (SEQ |
| | | ID NO: 26) |
| rs35478150_gDNA_R | Sanger sequencing | TCAACTTTGATTGCCAAGTGCA |
| | | (SEQ ID NO: 27) |
| rs35478150_cDNA_F | Sanger sequencing | GAGCAGTTCTGGAACCAACC (SEQ |
| | | ID NO: 28) |
| rs35478150_cDNA_R | Sanger sequencing | TTGAGGCCGAAGAGAGATGT (SEQ |
| | | ID NO: 29) |
| rs1808_cDNA_F | Sanger sequencing | CCTTGTGGAATTTGGGTCAT (SEQ |
| | | ID NO: 30) |
| rs1808_cDNA_R | Sanger sequencing | TCAAATGCAGGCACTTAGAAT (SEQ |
| | | ID NO: 31) |
| CDKL5_AmpSeqF | Amplicon | CAAGGAAAAAGAGAAGCAAGGA |
| | sequencing | (SEQ ID NO: 32) |
| CDKL5_AmpSeqR | Amplicon | ATTTTAATGGCTGGCTTTGG (SEQ |
| | sequencing | ID NO: 33) |
| CDKL5_BSS_F | Amplicon | TTTTAGTTTAGGTTGTTAGGGTTTG |
| | sequencing | (SEQ ID NO: 34) |
| CDKL5_BSS_R | Amplicon | TAAAAAAACACCTCAAATTTTACCC |
| | sequencing | (SEQ ID NO: 35) |
| CDKL5_ChIP_AF | ChIP-qPCR | TCATCCTCCTTGGAAACCCG (SEQ |
| | | ID NO: 36) |
| CDKL5_ChIP_AR | ChIP-qPCR | GTCATCGCCCAACCAGTACA (SEQ |
| | | ID NO: 37) |
| CDKL5_ChIP_BF | ChIP-qPCR | AGCAGCAGCAATGGACTTCG (SEQ |
| | | ID NO: 38) |
| CDKL5_ChIP_BR | ChIP-qPCR | AGAAATACAGGATGGAGGATGGT |
| | | (SEQ ID NO: 39) |
| CDKL5_ChIP_CF | ChIP-qPCR | AAGCGCTTCCTCCTCATTGG (SEQ |
| | | ID NO: 40) |
| CDKL5_ChIP_CR | ChIP-qPCR | AAAGCACCTCAGGTTTTGCC (SEQ |
| | | ID NO: 41) |
| MECP2_ChIP_F | ChIP-qPCR | AGCTGTTGATTGGCTGCTTT (SEQ |
| | | ID NO: 42) |
| MECP2_ChIP_R | ChIP-qPCR | TTCAAATTCCGCCCACTAAA (SEQ |
| | | ID NO: 43) |
| ChIP_SCML2F | ChIP-qPCR | CACCTCCCAGCTTCACTCTC (SEQ |
| | | ID NO: 44) |
| ChIP_SCML2R | ChIP-qPCR | CTGCGGGTTCATCTAGTTCC (SEQ |
| | | ID NO: 45) |
| CDKL5_common_F | ChIP-qPCR | ACAACCAGCATTCGATCCAT (SEQ |
| | | ID NO: 46) |
| CDKL5_A_allele_R | ChIP-qPCR | GCTGTCGGAATTGGGTACTGTTT |
| | | (SEQ ID NO: 47) |
| CDKL5_C_allele_R | ChIP-qPCR | GCTGTCGGAATTGGGTACTGTTG |
| | | (SEQ ID NO: 48) |
| CDKL5_qPCR_F | RT-qPCR | AACTCTTACTTGGCGCTCCC (SEQ |
| | | ID NO: 49) |
| CDKL5_qPCR_R | RT-qPCR | CTGTCCATCGCTAAGCTCCC (SEQ |
| | | ID NO: 50) |
| GAPDH_qPCR_F | RT-qPCR | AATCCCATCACCATCTTCCA (SEQ |
| | | ID NO: 51) |
| GAPDH_qPCR_R | RT-qPCR | CTCCATGGTGGTGAAGACG (SEQ ID |
| | | NO: 52) |

Informal Sequence Listing
SpdCas9 amino acid sequence
N-term half 1-713 aa
C-term half 713-1368 aa
(SEQ ID NO: 1)
DKKYSIGLAIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVSGQGDSLH
EHIANLAGSPAIKKGILQTVKVVDELVKVMGRHKPENIVI
EMARENQTTQKGQKNSRERMKRIEEGIKELGSQILKEHPV
ENTQLQNEKLYLYYLQNGRDMYVDQELDINRLSDYDVDAI
VPQSFLKDDSIDNKVLTRSDKNRGKSDNVPSEEVVKKMKN
YWRQLLNAKLITQRKFDNLTKAERGGLSELDKAGFIKRQL
VETRQITKHVAQILDSRMNTKYDENDKLIREVKVITLKSK
LVSDFRKDFQFYKVREINNYHHAHDAYLNAVVGTALIKKY
PKLESEFVYGDYKVYDVRKMIAKSEQEIGKATAKYFFYSN
IMNFFKTEITLANGEIRKRPLIETNGETGEIVWDKGRDFA
TVRKVLSMPQVNIVKKTEVQTGGFSKESILPKRNSDKLIA
RKKDWDPKKYGGFDSPTVAYSVLVVAKVEKGKSKKLKSVK
ELLGITIMERSSFEKNPIDFLEAKGYKEVKKDLIIKLPKY
SLFELENGRKRMLASAGELQKGNELALPSKYVNFLYLASH
YEKLKGSPEDNEQKQLFVEQHKHYLDEIIEQISEFSKRVI
LADANLDKVLSAYNKHRDKPIREQAENIIHLFTLTNLGAP
AAFKYFDTTIDRKRYTSTKEVLDATLIHQSITGLYETRID
LSQLGGD
RmaDnaB amino acid sequence
(SEQ ID NO: 2)
N-term break point 102 aa
C-term break point 52 aa
CLAGDTLITLADGRRVPIRELVSQQNFSVWALNPQTYRLE
RARVSRAFCTGIKPVYRLTTRLGRSIRATANHRFLTPQGW
KRVDELQPGDYLALPRRIPTASMAAACPELRQLAQSDVYW
DPIVSIEPDGVEEVEDLTVPGPHNFVANDIIAHN
Amino acid sequence for SpdCas9N-RmaDnaB
(SEQ ID NO: 3)
DKKYSIGLAIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVCLAGDTLI
TLADGRRVPIRELVSQQNESVWALNPQTYRLERARVSRAF
CTGIKPVYRLTTRLGRSIRATANHRFLTPQGWKRVDELQP
GDYLALPRRIPTAS
Amino acid sequence for RmaDnaB-SpdCas9C
(SEQ ID NO: 4)
MAAACPELRQLAQSDVYWDPIVSIEPDGVEEVEDLTVPGP
HNFVANDIIAHNSGQGDSLHEHIANLAGSPAIKKGILQTV
KVVDELVKVMGRHKPENIVIEMARENQTTQKGQKNSRERM
KRIEEGIKELGSQILKEHPVENTQLQNEKLYLYYLQNGRD
MYVDQELDINRLSDYDVDAIVPQSFLKDDSIDNKVLTRSD
KNRGKSDNVPSEEVVKKMKNYWRQLLNAKLITQRKFDNLT
KAERGGLSELDKAGFIKRQLVETRQITKHVAQILDSRMNT
KYDENDKLIREVKVITLKSKLVSDFRKDFQFYKVREINNY
HHAHDAYLNAVVGTALIKKYPKLESEFVYGDYKVYDVRKM
IAKSEQEIGKATAKYFFYSNIMNFFKTEITLANGEIRKRP
LIETNGETGEIVWDKGRDFATVRKVLSMPQVNIVKKTEVQ
TGGFSKESILPKRNSDKLIARKKDWDPKKYGGFDSPTVAY
SVLVVAKVEKGKSKKLKSVKELLGITIMERSSFEKNPIDF
LEAKGYKEVKKDLIIKLPKYSLFELENGRKRMLASAGELQ
KGNELALPSKYVNFLYLASHYEKLKGSPEDNEQKQLFVEQ
HKHYLDEIIEQISEFSKRVILADANLDKVLSAYNKHRDKP
IREQAENIIHLFTLTNLGAPAAFKYFDTTIDRKRYTSTKE
VLDATLIHQSITGLYETRIDLSQLGGDSRAD
dCas9-intein-all in one (underlined portions
are from dCas9 portein; bold portions are
from C-terminal split protein)
SEQ ID NO: 5
DKKYSIGLAIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNEDKNLPNEKVLPKHSLLYEYFTVY
NELTKVKYVTEGMRKPAFLSGEQKKAIVDLLFKTNRKVTV
KQLKEDYFKKIECFDSVEISGVEDRENASLGTYHDLLKII
KDKDFLDNEENEDILEDIVLTLTLFEDREMIEERLKTYAH
LEDDKVMKQLKRRRYTGWGRLSRKLINGIRDKQSGKTILD
FLKSDGFANRNFMQLIHDDSLTFKEDIQKAQVCLAGDTLI
TLADGRRVPIRELVSQQNFSVWALNPQTYRLERARVSRAF
CTGIKPVYRLTTRLGRSIRATANHRFLTPQGWKRVDELQP
GDYLALPRRIPTASMAAACPELRQLAQSDVYWDPIVSIEP
DGVEEVFDLTVPGPHNFVANDIIAHNSGQGDSLHEHIANL
AGSPAIKKGILQTVKVVDELVKVMGRHKPENIVIEMAREN
QTTQKGQKNSRERMKRIEEGIKELGSQILKEHPVENTQLQ
NEKLYLYYLQNGRDMYVDQELDINRLSDYDVDAIVPQSFL
KDDSIDNKVLTRSDKNRGKSDNVPSEEVVKKMKNYWRQLL
NAKLITQRKFDNLTKAERGGLSELDKAGFIKRQLVETRQI
TKHVAQILDSRMNTKYDENDKLIREVKVITLKSKLVSDFR
KDFQFYKVREINNYHHAHDAYLNAVVGTALIKKYPKLESE
FVYGDYKVYDVRKMIAKSEQEIGKATAKYFFYSNIMNFFK
TEITLANGEIRKRPLIETNGETGEIVWDKGRDFATVRKVL
SMPQVNIVKKTEVQTGGFSKESILPKRNSDKLIARKKDWD
PKKYGGFDSPTVAYSVLVVAKVEKGKSKKLKSVKELLGIT
IMERSSFEKNPIDFLEAKGYKEVKKDLIIKLPKYSLFELE
NGRKRMLASAGELQKGNELALPSKYVNFLYLASHYEKLKG
SPEDNEQKQLFVEQHKHYLDEIIEQISEFSKRVILADANL
DKVLSAYNKHRDKPIREQAENIIHLFTLTNLGAPAAFKYF
DTTIDRKRYTSTKEVLDATLIHQSITGLYETRIDLSQLGG
DSRAD
IDT human codon optimized SpdCas9 sequence
Demarcations
N-term 1-2136 bp
C-term 2137-4101 bp
(SEQ ID NO: 6)
gacaagaagtactccattgggctcgctatcggcacaaaca
gcgtcggctgggccgtcattacggacgagtacaaggtgcc
gagcaaaaaattcaaagttctgggcaataccgatcgccac
agcataaagaagaacctcattggcgccctcctgttcgact
ccggggagacggccgaagccacgcggctcaaaagaacagc
acggcgcagatatacccgcagaaagaatcggatctgctac
ctgcaggagatctttagtaatgagatggctaaggtggatg
actctttcttccataggctggaggagtcctttttggtgga
ggaggataaaaagcacgagcgccacccaatctttggcaat
atcgtggacgaggtggcgtaccatgaaaagtacccaacca
tatatcatctgaggaagaagcttgtagacagtactgataa
ggctgacttgcggttgatctatctcgcgctggcgcatatg
atcaaatttcggggacacttcctcatcgagggggacctga
acccagacaacagcgatgtcgacaaactctttatccaact
ggttcagacttacaatcagcttttcgaagagaacccgatc
aacgcatccggagttgacgccaaagcaatcctgagcgcta
ggctgtccaaatcccggcggctcgaaaacctcatcgcaca
gctccctggggagaagaagaacggcctgtttggtaatctt
atcgccctgtcactcgggctgacccccaactttaaatcta
acttcgacctggccgaagatgccaagcttcaactgagcaa
agacacctacgatgatgatctcgacaatctgctggcccag
atcggcgaccagtacgcagacctttttttggcggcaaaga
acctgtcagacgccattctgctgagtgatattctgcgagt
gaacacggagatcaccaaagctccgctgagcgctagtatg
atcaagcgctatgatgagcaccaccaagacttgactttgc
tgaaggcccttgtcagacagcaactgcctgagaagtacaa
ggaaattttcttcgatcagtctaaaaatggctacgccgga
tacattgacggcggagcaagccaggaggaattttacaaat
ttattaagcccatcttggaaaaaatggacggcaccgagga
gctgctggtaaagcttaacagagaagatctgttgcgcaaa
cagcgcactttcgacaatggaagcatcccccaccagattc
acctgggcgaactgcacgctatcctcaggcggcaagagga
tttctacccctttttgaaagataacagggaaaagattgag
aaaatcctcacatttcggataccctactatgtaggccccc
tcgcccggggaaattccagattcgcgtggatgactcgcaa
atcagaagagaccatcactccctggaacttcgaggaagtc
gtggataagggggcctctgcccagtccttcatcgaaagga
tgactaactttgataaaaatctgcctaacgaaaaggtgct
tcctaaacactctctgctgtacgagtacttcacagtttat
aacgagctcaccaaggtcaaatacgtcacagaagggatga
gaaagccagcattcctgtctggagagcagaagaaagctat
cgtggacctcctcttcaagacgaaccggaaagttaccgtg
aaacagctcaaagaagactatttcaaaaagattgaatgtt
tcgactctgttgaaatcagcggagtggaggatcgcttcaa
cgcatccctgggaacgtatcacgatctcctgaaaatcatt
aaagacaaggacttcctggacaatgaggagaacgaggaca
ttcttgaggacattgtcctcacccttacgttgtttgaaga
tagggagatgattgaagaacgcttgaaaacttacgctcat
ctcttcgacgacaaagtcatgaaacagctcaagaggcgcc
gatatacaggatgggggcggctgtcaagaaaactgatcaa
tgggatccgagacaagcagagtggaaagacaatcctggat
tttcttaagtccgatggatttgccaacaggaacttcatgc
agttgatccatgatgactctctcacctttaaggaggacat
ccagaaagcacaagtttctggccagggggacagtcttcac
gagcacatcgctaatcttgcaggtagcccagctatcaaaa
agggaatactgcagaccgttaaggtcgtggatgaactcgt
caaagtaatgggaaggcataagcccgagaatatcgttatc
gagatggcccgagagaaccaaactacccagaagggacaga
agaacagtagggaaaggatgaagaggattgaagagggtat
aaaagaactggggtcccaaatccttaaggaacacccagtt
gaaaacacccagcttcagaatgagaagctctacctgtact
acctgcagaacggcagggacatgtacgtggatcaggaact
ggacatcaatcggctctccgactacgacgtggatgctatc
gtgccccagtcttttctcaaagatgattctattgataata
aagtgttgacaagatccgataaaaatagagggaagagtga
taacgtcccctcagaagaagttgtcaagaaaatgaaaaat
tattggcggcagctgctgaacgccaaactgatcacacaac
ggaagttcgataatctgactaaggctgaacgaggtggcct
gtctgagttggataaagccggcttcatcaaaaggcagctt
gttgagacacgccagatcaccaagcacgtggcccaaattc
tcgattcacgcatgaacaccaagtacgatgaaaatgacaa
actgattcgagaggtgaaagttattactctgaagtctaag
ctggtctcagatttcagaaaggactttcagttttataagg
tgagagagatcaacaattaccaccatgcgcatgatgccta
cctgaatgcagtggtaggcactgcacttatcaaaaaatat
cccaagcttgaatctgaatttgtttacggagactataaag
tgtacgatgttaggaaaatgatcgcaaagtctgagcagga
aataggcaaggccaccgctaagtacttcttttacagcaat
attatgaattttttcaagaccgagattacactggccaatg
gagagattcggaagcgaccacttatcgaaacaaacggaga
aacaggagaaatcgtgtgggacaagggtagggatttcgcg
acagtccggaaggtcctgtccatgccgcaggtgaacatcg
ttaaaaagaccgaagtacagaccggaggcttctccaagga
aagtatcctcccgaaaaggaacagcgacaagctgatcgca
cgcaaaaaagattgggaccccaagaaatacggcggattcg
attctcctacagtcgcttacagtgtactggttgtggccaa
agtggagaaagggaagtctaaaaaactcaaaagcgtcaag
gaactgctgggcatcacaatcatggagcgatcaagcttcg
aaaaaaaccccatcgactttctcgaggcgaaaggatataa
agaggtcaaaaaagacctcatcattaagcttcccaagtac
tctctctttgagcttgaaaacggccggaaacgaatgctcg
ctagtgcgggcgagctgcagaaaggtaacgagctggcact
gccctctaaatacgttaatttcttgtatctggccagccac
tatgaaaagctcaaagggtctcccgaagataatgagcaga
agcagctgttcgtggaacaacacaaacactaccttgatga
gatcatcgagcaaataagcgaGttctccaaaagagtgatc
ctcgccgacgctaacctcgataaggtgctttctgcttaca
ataagcacagggataagcccatcagggagcaggcagaaaa
cattatccacttgtttactctgaccaacttgggcgcgcct
gcagccttcaagtacttcgacaccaccatagacagaaagc
ggtacacctctacaaaggaggtcctggacgccacactgat
tcatcagtcaattacggggctctatgaaacaagaatcgac
ctctctcagctcggtggagac
N-intein IDT codon optimized DNA
sequence (306 bp)
(SEQ ID NO: 7)
TGTCTTGCGGGGGATACTCTCATAACCCTTGCCGACGGGC
GGCGCGTACCTATTAGGGAGTTGGTGTCACAGCAGAACTT
CTCCGTTTGGGCACTTAACCCACAAACGTATCGCTTGGAG
CGGGCGCGGGTTAGTAGGGCCTTCTGTACAGGTATAAAAC
CcGTGTATAGGCTCACCACTAGGCTTGGCCGGTCCATCCG
AGCCACGGCGAACCATCGCTTTTTGACTCCACAAGGATGG
AAGAGGGTTGACGAATTGCAGCCTGGAGATTACCTGGCTC
TGCCCCGGCGCATACCTACGGCTAGT
C-intein IDT codon optimized sequence
(156 bp)
(SEQ ID NO: 8)
atggcggcGGCCTGCCCAGAGCTTCGCCAGCTGGCGCAAA
GCGACGTATATTGGGACCCAATAGTAAGCATAGAACCAGA
CGGTGTCGAAGAGGTGTTTGACCTGACGGTGCCAGGCCCT
CACAACTTCGTTGCTAACGACATCATAGCCCATAAT
CMV promoter sequence
(SEQ ID NO: 9)
ATACGCGTTGACATTGATTATTGACTAGTTATTAATAGTA
ATCAATTACGGGGTCATTAGTTCATAGCCCATATATGGAG
TTCCGCGTTACATAACTTACGGTAAATGGCCCGCCTGGCT
GACCGCCCAACGACCCCCGCCCATTGACGTCAATAATGAC
GTATGTTCCCATAGTAACGCCAATAGGGACTTTCCATTGA
CGTCAATGGGTGGAGTATTTACGGTAAACTGCCCACTTGG
CAGTACATCAAGTGTATCATATGCCAAGTACGCCCCCTAT
TGACGTCAATGACGGTAAATGGCCCGCCTGGCATTATGCC
CAGTACATGACCTTATGGGACTTTCCTACTTGGCAGTACA
TCTACGTATTAGTCATCGCTATTACCATGGTGATGCGGTT
TTGGCAGTACATCAATGGGCGTGGATAGCGGTTTGACTCA
CGGGGATTTCCAAGTCTCCACCCCATTGACGTCAATGGGA
GTTTGTTTTGGCACCAAAATCAACGGGACTTTCCAAAATG
TCGTAACAACTCCGCCCCATTGACGCAAATGGGCGGTAGG
CGTGTACGGTGGGAGGTCTATATAAGCAGAGCTCGTTTAG
TGAACCGTCAGATCGCCTGGAGACGCCATCCACGCTGTTT
TGACCTCCATAGAAGACACCGGGACCGATCCAGCCTCCGG
ACTCTAGAGGATCGAACCCTT
U6 promoter sequence
(SEQ ID NO: 10)
GAGGGCCTATTTCCCATGATTCCTTCATATTTGCATATAC
GATACAAGGCTGTTAGAGAGATAATTAGAATTAATTTGAC
TGTAAACACAAAGATATTAGTACAAAATACGTGACGTAGA
AAGTAATAATTTCTTGGGTAGTTTGCAGTTTTAAAATTAT
GTTTTAAAATGGACTATCATATGCTTACCGTAACTTGAAA
GTATTTCGATTTCTTGGCTTTATATATCTTGTGGAAAGGA
CGAAACACC
Amino acid sequence
(SEQ ID NO: 11)
DALDDFDLDML
Nucleotide sequence
(SEQ ID NO: 12)
AGAGCATCGGACCGAAGCGG
Nucleotide sequence
(SEQ ID NO: 13)
GGGGGAGAACATACTCGGGG
Nucleotide sequence
(SEQ ID NO: 14)
CCCAGGTTGCTAGGGCTTGG
Nucleotide sequence
(SEQ ID NO: 15)
ATCGCCTGAAACTTGTCCGG
Nucleotide sequence
(SEQ ID NO: 16)
CGAAAGGGTGTGAAAGAGGG
Nucleotide sequence
(SEQ ID NO: 17)
TGGGGAAGGTAAAGCGGCGA
Nucleotide sequence
(SEQ ID NO: 18)
AGAGCATCGGACCGAAGCGG
Nucleotide sequence
(SEQ ID NO: 19)
GGGGGAGAACATACTCGGGG
Nucleotide sequence
(SEQ ID NO: 20)
CCCAGGTTGCTAGGGCTTGG
Nucleotide sequence
(SEQ ID NO: 21)
ATCGCCTGAA ACTTGTCCGG
Nucleotide sequence
(SEQ ID NO: 22)
CGAAAGGGTGTGAAAGAGGG
Nucleotide sequence
(SEQ ID NO: 23)
TGGGGAAGGTAAAGCGGCGA
Nucleotide sequence
(SEQ ID NO: 24)
GCTTGAGCAATTTCGGACCC
Nucleotide sequence
(SEQ ID NO: 25)
TGTGTCTCTTGCTGGTACCG
Nucleotide sequence
(SEQ ID NO: 26)
TGAGCCTGTGCCAGAGGATA
Nucleotide sequence
(SEQ ID NO: 27)
TCAACTTTGATTGCCAAGTGCA
Nucleotide sequence
(SEQ ID NO: 28)
GAGCAGTTCTGGAACCAACC
Nucleotide sequence
(SEQ ID NO: 29)
TTGAGGCCGAAGAGAGATGT
Nucleotide sequence
(SEQ ID NO: 30)
CCTTGTGGAATTTGGGTCAT
Nucleotide sequence
(SEQ ID NO: 31)
TCAAATGCAGGCACTTAGAAT
Nucleotide sequence
(SEQ ID NO: 32)
CAAGGAAAAAGAGAAGCAAGGA
Nucleotide sequence
(SEQ ID NO: 33)
ATTTTAATGGCTGGCTTTGG
Nucleotide sequence
(SEQ ID NO: 34)
TTTTAGTTTAGGTTGTTAGGGTTTG
Nucleotide sequence
(SEQ ID NO: 35)
TAAAAAAACACCTCAAATTTTACCC
Nucleotide sequence
(SEQ ID NO: 36)
TCATCCTCCTTGGAAACCCG
Nucleotide sequence
(SEQ ID NO: 37)
GTCATCGCCCAACCAGTACA
Nucleotide sequence
(SEQ ID NO: 38)
AGCAGCAGCAATGGACTTCG
Nucleotide sequence
(SEQ ID NO: 39)
AGAAATACAGGATGGAGGATGGT
Nucleotide sequence
(SEQ ID NO: 40)
AAGCGCTTCCTCCTCATTGG
Nucleotide sequence
(SEQ ID NO: 41)
AAAGCACCTCAGGTTTTGCC
Nucleotide sequence
(SEQ ID NO: 42)
AGCTGTTGATTGGCTGCTTT
Nucleotide sequence
(SEQ ID NO: 43)
TTCAAATTCCGCCCACTAAA
Nucleotide sequence
(SEQ ID NO: 44)
CACCTCCCAGCTTCACTCTC
Nucleotide sequence
(SEQ ID NO: 45)
CTGCGGGTTCATCTAGTTCC
Nucleotide sequence
(SEQ ID NO: 46)
ACAACCAGCATTCGATCCAT
Nucleotide sequence
(SEQ ID NO: 47)
GCTGTCGGAATTGGGTACTGTTT
Nucleotide sequence
(SEQ ID NO: 48)
GCTGTCGGAATTGGGTACTGTTG
Nucleotide sequence
(SEQ ID NO: 49)
AACTCTTACTTGGCGCTCCC
Nucleotide sequence
(SEQ ID NO: 50)
CTGTCCATCGCTAAGCTCCC
Nucleotide sequence
(SEQ ID NO: 51)
AATCCCATCACCATCTTCCA
Nucleotide sequence
(SEQ ID NO: 52)
CTCCATGGTGGTGAAGACG

1. An epigenetic editing system comprising:
(i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and
(ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an epigenetic modifier.

2. The system of claim 1, further comprising a third expression cassette comprising a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of a target gene transcription start site.

3. The system of claim 1, wherein the first and/or second expression cassettes further comprise a third polynucleotide sequence encoding a small guide RNA (sgRNA), which comprises a scaffold region and a spacer region, wherein the spacer region hybridizes to a sequence complementary to a target sequence adjacent to the 5′ end of a protospacer adjacent motif (PAM), with both the target sequence and the PAM located within 1 kilobase (kb) of the target gene transcription start site.

4. The system of claim 1, wherein the transcription activator is VP64, an MS2-loop SAM system, a mini-VPR, p30000RE, or any combination thereof.

5. The system of claim 1, wherein the dCas9 protein is a Streptococcus pyogenes dCas9 (spdCas9) protein.

6. The system of claim 5, wherein the N-dCas9 and C-dCas9 consist of the 1 to 713 segment and the 713 to 1368 segment of SEQ ID NO:1, respectively.

7. The system of claim 1, wherein the intein is Rhodothermus marinus (Rma) DNA helicase DnaB.

8. The system of claim 7, wherein the N-intein and C-intein consist of the 1 to 102 segment and the 103 to 154 segment of SEQ ID NO:2, respectively.

9. The system of claim 1, wherein the epigenetic modifier is a human Ten-Eleven Translocation methylcytosine dioxygenase 1 catalytic domain (hTET1CD), a Suntag, a DOT1 L catalytic domain, PRDM9CD, an amoeba Tet1 (NgTet1), or any combination thereof.

10. The system of claim 1, wherein each of the first, second, or third polynucleotide sequence is operably linked to a promoter and optionally further to a polyA sequence.

11. The system of claim 10, wherein the promoter is a CMV promoter.

12. The system of claim 1, wherein the N-terminal split protein further comprises at least one nuclear localization signal (NLS) located at the N-terminus to the transcription activator.

13. The system of claim 1, wherein the C-terminal split protein further comprises at least one NLS, preferably two or three NLS, located between the C-dCas9 and the epigenetic modifier.

14. (canceled)

15. The system of claim 1, wherein the first, second, or third expression cassette comprises a coding sequence encoding two or three sgRNAs.

16. The system of claim 2, wherein the first, second, and third expression cassettes are present in three separate vectors.

17. The system of claim 16, wherein each of the vectors is a viral vector or a plasmid.

18. The system of claim 17, wherein the viral vector is a lentiviral vector, an adeno-associated viral (AAV) vector, or an adenoviral vector.

19. The system of claim 2, wherein the target gene is CDKL5.

20. The system of claim 2, wherein the target sequence comprises or consists of AGAGCATCGGACCGAAGCGG (SEQ ID NO:12), GGGGGAGAACATACTCGGGG (SEQ ID NO:13), or CCCAGGTTGCTAGGGCTTGG (SEQ ID NO:14).

21-47. (canceled)

48. The system of claim 12, wherein the NLS is an SV40 NLS.

49. The system of claim 13, wherein the NLS is an SV40 NLS.

50. A composition comprising the system of claim 1, optionally with a pharmaceutically acceptable carrier.


SEQUENCE LISTING (52 sequences)

SEQ ID NO: 1 - protein, 1367 aa, synthetic construct
  source 1..1367: note = SpdCas9 amino acid sequence
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 154 aa, synthetic construct
  source 1..154: note = RmaDnaB amino acid sequence
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASMAAACPELRQLAQSDVYWDPIVSIEPDGVEEVFDLTVPGPHNFVANDIIAHN

SEQ ID NO: 3 - protein, 814 aa, synthetic construct
  source 1..814: note = Amino acid sequence for SpdCas9N-RmaDnaB
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 4 - protein, 711 aa, synthetic construct
  source 1..711: note = Amino acid sequence for RmaDnaB-SpdCas9C
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 5 - protein, 1525 aa, synthetic construct
  source 1..1525: note = dCas9-intein-all in one
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASMAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 6 - DNA, 4101 bp, synthetic construct
  source 1..4101: mol_type = other DNA; note = IDT human codon optimized SpdCas9 sequence
gacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaacaggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgagttctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 7 - DNA, 306 bp, synthetic construct
  source 1..306: mol_type = other DNA; note = N-intein IDT codon optimized DNA sequence (306bp)
tgtcttgcgggggatactctcataacccttgccgacgggcggcgcgtacctattagggagttggtgtcacagcagaacttctccgtttgggcacttaacccacaaacgtatcgcttggagcgggcgcgggttagtagggccttctgtacaggtataaaacccgtgtataggctcaccactaggcttggccggtccatccgagccacggcgaaccatcgctttttgactccacaaggatggaagagggttgacgaattgcagcctggagattacctggctctgccccggcgcatacctacggctagt

SEQ ID NO: 8 - DNA, 156 bp, synthetic construct
  source 1..156: mol_type = other DNA; note = C-intein IDT codon optimized sequence (156bp)
atggcggcggcctgcccagagcttcgccagctggcgcaaagcgacgtatattgggacccaatagtaagcatagaaccagacggtgtcgaagaggtgtttgacctgacggtgccaggccctcacaacttcgttgctaacgacatcatagcccataat

SEQ ID NO: 9 - DNA, 701 bp, synthetic construct
  source 1..701: mol_type = other DNA; note = CMV promoter sequence
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaaccctt

SEQ ID NO: 10 - DNA, 249 bp, synthetic construct
  source 1..249: mol_type = other DNA; note = U6 promoter sequence
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 11 - protein, 11 aa, synthetic construct
  source 1..11: note = Tetrameric repeat sequence
DALDDFDLDML

SEQ ID NO: 12 - DNA, 20 bp, synthetic construct
  source 1..20: mol_type = other DNA; note = Nucleotide target sequence
agagcatcggaccgaagcgg

SEQ ID NO: 13 - DNA, 20 bp, synthetic construct
  source 1..20: mol_type = other DNA; note = Nucleotide target sequence
gggggagaacatactcgggg

SEQ ID NO: 14 - DNA, 20 bp, synthetic construct
  source 1..20: mol_type = other DNA; note = Nucleotide target sequence
cccaggttgctagggcttgg

SEQ ID NO: 15 - DNA, 20 bp, synthetic construct
  source 1..20: mol_type = other DNA; note = Nucleotide target sequence
atcgcctgaaacttgtccgg

SEQ ID NO: 16 - DNA, 20 bp, synthetic construct
  source 1..20: mol_type = other DNA; note = Nucleotide target sequence
cgaaagggtgtgaaagaggg

SEQ ID NO: 17 - DNA, 20 bp, synthetic construct
  source 1..20: mol_type = other DNA; note = Nucleotide target sequence
tggggaaggtaaagcggcga

SEQ ID NO: 18 - DNA, 20 bp, synthetic construct
  source 1..20: mol_type = other DNA; note = Nucleotide target sequence
agagcatcggaccgaagcgg

SEQ ID NO: 19 - DNA, 20 bp, synthetic construct
  source 1..20: mol_type = other DNA; note = Nucleotide target sequence
gggggagaacatactcgggg

SEQ ID NO: 20 - DNA, 20 bp, synthetic construct
  misc_feature 1..20: note = Description of sequence:
  source 1..20: mol_type = other DNA; note = Nucleotide target sequence
cccaggttgctagggcttgg

SEQ ID NO: 21 - DNA, 20 bp, synthetic construct
  source 1..20: mol_type = other DNA; note = Nucleotide target sequence
atcgcctgaaacttgtccgg

SEQ ID NO: 22 - DNA, 20 bp, synthetic construct
  source 1..20: mol_type = other DNA; note = Nucleotide target sequence
cgaaagggtgtgaaagaggg

SEQ ID NO: 23 - DNA, 20 bp, synthetic construct
  source 1..20: mol_type = other DNA; note = Nucleotide target sequence
tggggaaggtaaagcggcga

SEQ ID NO: 24 - DNA, 20 bp, synthetic construct
  source 1..20: mol_type = other DNA; note = Nucleotide target sequence
gcttgagcaatttcggaccc

SEQ ID NO: 25 - DNA, 20 bp, synthetic construct
  source 1..20: mol_type = other DNA; note = Nucleotide target sequence
tgtgtctcttgctggtaccg

SEQ ID NO: 26 - DNA, 20 bp, synthetic construct
  source 1..20: mol_type = other DNA; note = Nucleotide target sequence
tgagcctgtgccagaggata

SEQ ID NO: 27 - DNA, 22 bp, synthetic construct
  source 1..22: mol_type = other DNA; note = Nucleotide target sequence
tcaactttgattgccaagtgca

SEQ ID NO: 28 - DNA, 20 bp, synthetic construct
  source 1..20: mol_type = other DNA; note = Nucleotide target sequence
gagcagttctggaaccaacc

SEQ ID NO: 29 - DNA, 20 bp, synthetic construct
  source 1..20: mol_type = other DNA; note = Nucleotide target sequence
ttgaggccgaagagagatgt

SEQ ID NO: 30 - DNA, 20 bp, synthetic construct
  source 1..20: mol_type = other DNA; note = Nucleotide target sequence
ccttgtggaatttgggtcat

SEQ ID NO: 31 - DNA, 21 bp, synthetic construct
  source 1..21: mol_type = other DNA; note = Nucleotide target sequence
tcaaatgcaggcacttagaat

SEQ ID NO: 32 - DNA, 22 bp, synthetic construct
  source 1..22: mol_type = other DNA; note = Nucleotide target sequence
caaggaaaaagagaagcaagga

SEQ ID NO: 33 - DNA, 20 bp, synthetic construct
  source 1..20: mol_type = other DNA; note = Nucleotide target sequence
attttaatggctggctttgg

SEQ ID NO: 34 - DNA, 25 bp, synthetic construct
  source 1..25: mol_type = other DNA; note = Nucleotide target sequence
ttttagtttaggttgttagggtttg

SEQ ID NO: 35 - DNA, 25 bp, synthetic construct
  source 1..25: mol_type = other DNA; note = Nucleotide target sequence
taaaaaaacacctcaaattttaccc

SEQ ID NO: 36 - DNA, 20 bp, synthetic construct
  source 1..20: mol_type = other DNA; note = Nucleotide target sequence
tcatcctccttggaaacccg

SEQ ID NO: 37 - DNA, 20 bp, synthetic construct
  source 1..20: mol_type = other DNA; note = Nucleotide target sequence
gtcatcgcccaaccagtaca

SEQ ID NO: 38 - DNA, 20 bp, synthetic construct
  source 1..20: mol_type = other DNA; note = Nucleotide target sequence
agcagcagcaatggacttcg

SEQ ID NO: 39 - DNA, 23 bp, synthetic construct
  source 1..23: mol_type = other DNA; note = Nucleotide target sequence
agaaatacaggatggaggatggt

SEQ ID NO: 40 - DNA, 20 bp, synthetic construct
  source 1..20: mol_type = other DNA; note = Nucleotide target sequence
aagcgcttcctcctcattgg

SEQ ID NO: 41 - DNA, 20 bp, synthetic construct
  source 1..20: mol_type = other DNA; note = Nucleotide target sequence
aaagcacctcaggttttgcc

SEQ ID NO: 42 - DNA, 20 bp, synthetic construct
  source 1..20: mol_type = other DNA; note = Nucleotide target sequence
agctgttgattggctgcttt

SEQ ID NO: 43 - DNA, 20 bp, synthetic construct
  source 1..20: mol_type = other DNA; note = Nucleotide target sequence
ttcaaattccgcccactaaa

SEQ ID NO: 44 - DNA, 20 bp, synthetic construct
  source 1..20: mol_type = other DNA; note = Nucleotide target sequence
cacctcccagcttcactctc

SEQ ID NO: 45 - DNA, 20 bp, synthetic construct
  source 1..20: mol_type = other DNA; note = Nucleotide target sequence
ctgcgggttcatctagttcc

SEQ ID NO: 46 - DNA, 20 bp, synthetic construct
  source 1..20: mol_type = other DNA; note = Nucleotide target sequence
acaaccagcattcgatccat

SEQ ID NO: 47 - DNA, 23 bp, synthetic construct
  source 1..23: mol_type = other DNA; note = Nucleotide target sequence
gctgtcggaattgggtactgttt

SEQ ID NO: 48 - DNA, 23 bp, synthetic construct
  source 1..23: mol_type = other DNA; note = Nucleotide target sequence
gctgtcggaattgggtactgttg

SEQ ID NO: 49 - DNA, 20 bp, synthetic construct
  source 1..20: mol_type = other DNA; note = Nucleotide target sequence
aactcttacttggcgctccc

SEQ ID NO: 50 - DNA, 20 bp, synthetic construct
  source 1..20: mol_type = other DNA; note = Nucleotide target sequence
ctgtccatcgctaagctccc

SEQ ID NO: 51 - DNA, 20 bp, synthetic construct
  source 1..20: mol_type = other DNA; note = Nucleotide target sequence
aatcccatcaccatcttcca

SEQ ID NO: 52 - DNA, 19 bp, synthetic construct
  source 1..19: mol_type = other DNA
ctccatggtggtgaagacg